(19) United States
(12) Patent Application Publication
(10) Pub. No.: US 2010/0137358 A1
(43) Pub. Date: Jun. 3, 2010

(54) SOLIFENACIN COMPOSITIONS

(75) Inventors: Pramod Kharwade, Chhindwara (IN); Movva Snehalatha, Hyderabad (IN); Atul Vishvanath Patil, Karnataka (IN); Narayanan Badri Vishwanathan, Chennai (IN); Indu Bhushan, Hyderabad (IN); Venkata Nookaraju Sreedharala, Hyderabad (IN); Harshal Prabhakar Bhagwatwar, Hyderabad (IN); Surya Narayana Devarakonda, Hyderabad (IN); Ravi Kumar Komareddy, Hyderabad (IN); Azeezulla Baig Mohammed, Vijayawada (IN); Arjun Kumar Tummala, Guntur (IN); Jaydeepkumar Dahyabhai Lilakar, Valsad (IN); Srirami Reddy Kikkuru, Guntur (IN); Swarupa Dudipala, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500016 Andhra Pradesh (IN); DR. REDDY'S LABORATORIES, INC., Bridgewater, NJ (US)

(21) Appl. No.: 12/595,290

(22) PCT Filed: Apr. 11, 2008
(86) PCT No.: PCT/US08/60010

§ 371 Date: Oct. 9, 2009

Related U.S. Application Data
(60) Provisional application No. 60/957,235, filed on Aug. 22, 2007.
(60) Provisional application No. 61/025,863, filed on Feb. 4, 2008.
(60) Provisional application No. 60/030,374, filed on Nov. 5, 1996.

(30) Foreign Application Priority Data
Apr. 11, 2007 (IN) 769/CHE/2007
May 23, 2007 (IN) 1084/CHE/2007
Oct. 4, 2007 (IN) 2237/CHE/2007

(51) Int. Cl. A61K 31/4725; C07D 453/04; A61P 13/10

(52) U.S. Cl. 514/305; 546/134

(57) ABSTRACT

Compositions and/or formulations comprising solifenacin or a salt thereof and processes for preparing the same. Certain compositions and formulations contain a stable amorphous form of solifenacin succinate.

The present invention relates to solifenacin or salts thereof, and processes for preparing the same. Also the invention relates to stable solifenacin succinate and processes for preparing the same. The invention further relates to compositions and their pharmaceutical formulations, which comprise amorphous solifenacin succinate, and processes for preparing the same. And further the invention includes stable compositions and their formulations comprising amorphous solifenacin succinate, and processes for preparing the same. The invention also relates to crystalline solifenacin succinate substantially free of amorphous solifenacin succinate and its compositions and/or formulations, processes for preparing the same.

Solifenacin succinate is a muscarinic receptor antagonist. Solifenacin succinate has a chemical name butanedioic acid, compound with 1(S)-3(R)-1-azabicyclo[2.2.2]oct-3-yl 3,4-dihydro-1-phenyl-2(1H)-isoquinolinecarboxylate (1:1) having an empirical formula C₂₃H₂₆N₂O₂C₄H₆O₄ and a molecular weight of 480.55. The structural formula for solifenacin succinate is Formula 1.

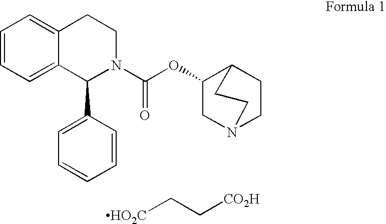

Solifenacin succinate is a white to pale yellowish white crystal or crystalline powder. It is freely soluble at room temperature in water, glacial acetic acid, dimethyl sulfoxide, and methanol.

Solifenacin succinate is available in the U.S. market from Astellas Pharmaceuticals Inc. under the name VESIcare®, in two strengths, 5 mg and 10 mg of solifenacin succinate, and formulated as tablets for oral administration. In addition to the active ingredient solifenacin succinate, each VESIcare tablet also contains the following inactive excipients: lactose monohydrate, corn starch, hypromellose 2910, magnesium stearate, talc, polyethylene glycol 8000 and titanium dioxide with yellow ferric oxide (5 mg) or red ferric oxide (10 mg).

Solifenacin succinate is approved for the treatment of overactive bladder with symptoms of urge urinary incontinence, urgency and urinary frequency.

U.S. Pat. No. 6,017,927 discloses solifenacin and a process to prepare the same. International Application Publication No. WO 2006/070735 describes stable granular pharmaceutical compositions of solifenacin or its salts.

International Application Publication Nos. WO 2005/092889 A1 AND WO 2006/090759, and European Patent Application Nos. 1728791, 1726304, 1714965, and 1832288 disclose pharmaceutical compositions of solifenacin or salts thereof.

Generally it is known that drugs or drug products, when exposed to different environmental conditions, are prone to different reactions, which may cause drug to degrade and generate impurities. In addition to this during manufacturing process, the drug or drug product may also be subjected to attrition/pressure such as during mixing, granulation, drying, milling, etc. Due to this the drug may loose its crystalline nature and may be converted into other forms such as amorphous form or other crystalline forms, which may be unstable and generate impurities. Hence it becomes difficult to maintain the drug in crystalline form.

From the literature, it is known that solifenacin and its derivatives are stable in the crystalline form, and unstable in the amorphous form. European Patent Application No. 1728791 A1 describes attempts that have been made to obtain stable formulations of solifenacin or its salts. One of the attempts was to control the amorphous content of solifenacin or its salts for use in stable solid formulations and it has been reported that if the amorphous content is 77% or less, then degradation over time, i.e., temporal decomposition, could be inhibited. Other attempts made to inhibit temporal decomposition were to maintain low moisture content in a drug product during manufacturing processing, stabilizing the drug product with polyethylene glycol (macrogol), etc.

When the active substance solifenacin succinate is unstable during the process of preparing compositions, there arises a need for stabilized amorphous solifenacin succinate, which retains its stability even in the compositions and/or formulations so as to maintain the degradation products or impurities within regulatory acceptable limits to minimize the possibility of some adverse influence on therapeutic effects.

By the processes of the present invention, solifenacin succinate in amorphous or crystalline form can be obtained, which is stable. Further, in the processes of preparing the compositions of the present invention, the crystalline form of solifenacin succinate may be maintained in crystalline form in the compositions.

It is further known that solifenacin or its salts has exceedingly high solubility and exceedingly strong bitterness and astringency in relation to a variety of solvents. Therefore, there is a further need to develop compositions with a high level of convenience, which can mask the bitterness, and astringency of the pharmaceutical ingredient

These and other needs are addressed by the present invention.

### SUMMARY OF THE INVENTION

The present invention relates to solifenacin succinate and processes for preparing the same. Further, the invention relates to compositions and/or formulations comprising stable solifenacin succinate and processes for preparing the same.

In embodiments the invention includes stable amorphous solifenacin succinate and process for preparing the same.

In an aspect, the present invention includes compositions and/or formulations comprising stable amorphous form of solifenacin succinate.

In an aspect the invention includes processes to prepare amorphous solifenacin succinate, wherein an embodiment of a process comprises:

1) dissolving solifenacin succinate in a suitable solvent;

2) optionally, filtering a solution; and

3) removing the solvent.

In an embodiment the invention includes substantially amorphous solifenacin succinate and processes for preparing the same.

Further, the invention also includes compositions and/or formulations comprising substantially amorphous solifenacin succinate and processes for preparing the same.

In an embodiment the invention includes stable compositions and/or formulations comprising substantially amorphous solifenacin succinate.

In embodiments the invention includes stable compositions and/or formulations comprising solifenacin succinate and at least one pharmaceutical acceptable carrier such as a resin, cyclodextrin or its derivatives, polyvinyl pyrrolidone, cellulose or its derivatives, dibasic calcium phosphate, propylene glycol, or combinations thereof.

In an aspect, the invention includes stable compositions comprising solifenacin succinate and at least one carrier, in the form of premix compositions.

In an aspect, the invention includes processes for preparing solid premix compositions comprising solifenacin succinate and at least one carrier, wherein an embodiment of a process comprises:

1) providing a solution or dispersion comprising dissolved solifenacin or a salt thereof and at least one pharmaceutical carrier;

2) optionally, filtering a solution; and

3) removing the solvent to recover a stable premix comprising solifenacin or a salt thereof.

In embodiments the invention includes stable premix compositions comprising solifenacin succinate and at least one antioxidant.

In embodiments the invention includes weight ratios of solifenacin or its salts to antioxidant in the range of about 1:0.001 to 1:1, or from about 1:0.001 to about 1:0.1.

In an embodiment the invention includes stable premix compositions of solifenacin succinate, at least one pharmaceutically acceptable carrier and at least one antioxidant.

In other embodiments the invention includes stable formulations comprising solifenacin succinate and at least one antioxidant.

Further, the invention includes crystalline solifenacin succinate substantially free of amorphous solifenacin succinate and processes to prepare the same.

In another embodiment the invention includes compositions and/or formulations comprising solifenacin succinate, amorphous solifenacin succinate, or substantially amorphous solifenacin succinate, wherein compositions and/or formulations substantially retain the XRD pattern of the starting solifenacin succinate material.

In embodiments the invention includes compositions and/or formulations comprising solifenacin succinate wherein the compositions and/or formulations mask the bitter taste of solifenacin succinate.

In other embodiments the invention includes processes to prepare compositions and/or formulations comprising solifenacin succinate, such that solifenacin succinate remains substantially unchanged in polymorphic form during processing.

In embodiments the invention includes methods of treating overactive bladder with symptoms of urge urinary incontinence, urgency and urinary frequency, using stable compositions and/or formulations of the present invention.

Aspects of the invention provide a process for preparing stabilized solifenacin or a salt thereof, comprising:

(a) providing a solution containing solifenacin or a salt thereof, a pharmaceutically acceptable carrier, and optionally an antioxidant, then removing solvent or adding an anti-solvent to precipitate a solid; or

(b) providing a solution containing solifenacin or a salt thereof in a nonvolatile solvent, and optionally adsorbing the solution onto a solid pharmaceutical excipient; or

(c) providing a solution containing solifenacin or a salt thereof, and contacting the solution with an insoluble resin to form a resinate; or

(d) providing a solution containing solifenacin or a salt thereof and a cyclodextrin, and combining the solution with a solid pharmaceutical excipient.

Other aspects of the invention provide a solid premix composition prepared by combining a solution comprising solifenacin succinate and an organic solvent with a pharmaceutically acceptable carrier, and removing solvent.

### BRIEF DESCRIPTION OF THE DRAWINGS

FIG. 1 is a X-ray powder diffraction (“XRD”) pattern for the crystalline solifenacin succinate prepared according to Example 1.

FIG. 2 is a near infrared (“NIR”) absorption spectrum for the amorphous solifenacin succinate prepared according to Example 2.

FIG. 3 is a XRD pattern for the amorphous solifenacin succinate prepared according to Example 2.

FIG. 4 is a XRD pattern for the amorphous solifenacin succinate prepared according to Example 2, after 7 days storage in a triple laminated package under a nitrogen atmosphere.

FIG. 5 is a XRD pattern for composition prepared according to Example 6B, after storage at ambient temperature for 6 months.

FIG. 6 shows comparative XRD patterns of a physical mixture of crystalline solifenacin succinate and Amberlite™ IRP 88 (A), a composition prepared according to Example 9 (B), a similarly prepared composition after exposure to 40° C. and 75% relative humidity (“RH”) for three months (C), and a similarly prepared composition but without solifenacin succinate (D).

FIG. 7 is a XRD pattern for tablets prepared according to Example 10.

FIG. 8 is a XRD pattern for tablets prepared according to Example 12.

FIG. 9 is a XRD pattern for tablets prepared according to Example 13.

FIG. 10 shows comparative XRD patterns for the composition prepared according to Example 14 after exposure to 40° C. and 75% RH for 3 months (A), the initial composition before exposure (B), and crystalline solifenacin succinate (C).

FIG. 11 shows comparative XRD patterns for the composition prepared according to Example 15 after exposure to 40° C. and 75% RH for 3 months (A), and a similarly prepared composition but without solifenacin succinate (B).

### DETAILED DESCRIPTION OF THE INVENTION

The present invention relates to solifenacin succinate and processes for preparing the same. The invention further relates to compositions and/or formulations comprising solifenacin succinate and processes for preparing the same.

The invention also relates to substantially amorphous solifenacin succinate and processes for preparing the same. Also the invention relates to compositions and/or formulations comprising substantially amorphous solifenacin succinate and processes for preparing the same.

Further the invention relates to stable amorphous solifenacin succinate and processes for preparing the same. The invention also relates to stable compositions and/or formulations comprising stable amorphous solifenacin succinate and processes for preparing the same.

The invention further relates to crystalline solifenacin succinate substantially free of amorphous solifenacin succinate and processes for preparing the same. The invention also relates to compositions and/or formulations comprising crystalline solifenacin succinate substantially free of amorphous solifenacin succinate and processes for preparing the same.

The term “amorphous solifenacin succinate” or “substantially amorphous solifenacin succinate” in the present invention refers to solifenacin succinate, which can have some crystalline content and which has at least: about 80%; about 90%; about 95%; or about 99%; by weight of amorphous compound.

The term “crystalline solifenacin succinate” in the present invention refers to solifenacin succinate, which can have some amorphous content and which has not more than: about 20%; about 10%; about 5%; or about 1%; by weight of amorphous compound.

The term “composition” in the present invention refers to solid premix compositions comprising solifenacin succinate, either in crystalline or amorphous form, and a solid carrier for use in preparing solid pharmaceutical formulations with no specific limitations, wherein solifenacin succinate is in combination with at least one pharmaceutical acceptable carrier such as a cyclodextrin or a derivative thereof, a resin, dibasic calcium phosphate (e.g., Fujicalin®), povidone, a cellulose derivative, or any combination thereof.

The term “formulation” refers to pharmaceutical dosage forms containing compositions comprising solifenacin or its derivatives. The pharmaceutical formulations of the present invention can be prepared as solid oral dosage forms or liquid dosage forms. The solid forms include for example tablets, caplets, capsules (hard or soft gelatin capsules), oral disintegrating dosage forms, chewable dosage forms, pills, granules, sachets and the like. The liquid forms include for example solutions, syrups, suspensions or dispersions, or emulsions like micro-emulsions or multiple-emulsions; elixirs and so on.

The term “derivative” of solifenacin includes solifenacin base or salts, enantiomers, analogs, hydrates, solvates, polymorphs, prodrugs, esters, amides, or active metabolites thereof.

Various pharmaceutically acceptable salts of solifenacin include but are not limited to acid addition salts with a mineral acid including, without limitation, hydrochloric acid, hydrobromic acid, hydroiodic acid, sulfuric acid, nitric acid or phosphoric acid, etc. Pharmaceutically acceptable salts also include salts with an organic acid, for example but not limited to formic acid, acetic acid, propionic acid, oxalic acid, malonic acid, succinic acid, fumaric acid, maleic acid, lactic acid, malic acid, citric acid, tartaric acid, carbonic acid, picric acid, methanesulfonic acid, ethanesulfonic acid, glutamic acid, etc.

The degradation of a drug substance in a pharmaceutical formulation can involve, for example, oxidation or reduction reactions, hydrolysis reactions, racemization, photodegradation and polymeric degradation. It has been described that these reactions have a correlation with exposure to heat, oxygen, light, water, etc., and interactions with other components of the formulation. As described above, numerous causes of drug degradation should be considered so as to obtain stable drug products.

During process of manufacturing, the crystalline content of active substances such as solifenacin succinate may be reduced via crystal deformation. This may be due to kneading, etc. during production steps, and pressure, abrasion, heat and the like imposed during granulation or pressure molding processes. Because of this, it is difficult to maintain the amorphous content within the specified ranges during process of preparing the compositions, or in other words the amorphous content of the drug generated in the manufacturing process cannot be predicted.

Even maintaining the amorphous content in the formulation within the specified ranges requires specialized equipment and careful scheduling of the preparation, which increases the cost of the process in terms of stabilizers, equipment to be used, conditions to be maintained, packaging material used to store the drug, its compositions, or its formulations, etc.

Therapeutic agents for treating pollakiuria and incontinence of urine, such as solifenacin succinate, are administered for a long period of time. Therefore, the active substance should be of high purity with minimum levels of degradation products or impurities to avoid unwanted adverse effects.

Surprisingly, and contrary to teachings in the art, it has been found that solifenacin succinate may be made stable when it is in either in amorphous or in substantially amorphous form. Also, the compositions formed by using this amorphous or substantially amorphous solifenacin succinate have been found to be stable against polymorphic changes, as well as having chemical stability.

In embodiments the invention relates to amorphous solifenacin succinate and processes to prepare the same.

In other embodiments the present invention further includes substantially amorphous solifenacin succinate and processes to prepare the same.

In other embodiments the invention includes stable amorphous solifenacin succinate and processes to prepare the same.

Substantially amorphous solifenacin succinate in the present invention includes more than about 80%, more than about 90%, more than about 95%, or more than about 99%, by weight of amorphous content.

In an aspect, the present invention provides processes for the preparation of amorphous solifenacin or salts thereof, wherein an embodiment of a process comprises:

a) providing a solution of solifenacin or its pharmaceutically acceptable salt in a organic solvent; and

b) removing the solvent to recover amorphous solifenacin or its salt.

The providing step a) may involve dissolving the active substance in a solvent that is suitable for easy, commercially viable solvent removal via distillation, etc. in step b).

A solution of solifenacin succinate may be obtained by dissolving solifenacin, or a salt such as the succinate, in a suitable solvent. The solvent that can be used for preparing amorphous solifenacin succinate may be any organic solvent from the various classes of solvents such as for example alcohols, ketones, esters, ethers, halogenated hydrocarbons, aromatic hydrocarbons such as toluene, xylene, chlorobenzene, etc, nitriles, aprotic polar solvents, or mixtures of any two or more thereof. Alcohol solvents include for example methanol, ethanol, denatured spirits, n-propanol, isopropanol, n-butanol, isobutanol, and t-butanol and the like. Ketone solvents include acetone, propanone, 2-butanone and the like. Halogenated hydrocarbons include for example dichloromethane, 1,2-dichloroethane, chloroform, carbon tetrachloride and the like. Ester solvents include for example ethyl acetate, n-propyl acetate, isopropyl acetate, n-butyl acetate, tertiary-butyl acetate and the like. Ether solvents include for example dimethyl ether, diethyl ether, methyl tertiary-butyl ether, ethyl methyl ether, diisopropyl ether, tetrahydrofuran, dioxane and the like. The hydrocarbon may be any solvent from this class such as for example toluene, xylene and the like. The nitrile solvents may include acetonitrile, propionitrile and the like. Aprotic polar solvents include N,N-dimethylformide (DMF), dimethylsulfoxide (DMSO), N,N-dimethylacetamide (DMA) and the like. Acidic solvents include formic acid, acetic acid and the like. This listing is not intended to be exhaustive, and combinations of solvents that are useful can include more than one member of a class, and/or can be from different classes.

These and other classes of solvents known to a person skilled in the art are all contemplated without limitation. The organic solvent acceptable for the practice of the process described herein preferably provide sufficient solubility for the active substance, and do not cause any undesirable chemical reactions with the solifenacin or salt, such as degradation, under the conditions of processing.

The dissolution temperatures can range from about 0° C. to about 70° C., or the reflux temperature of the solvent used.

The recovering step b) may involve removing the solvent by distillation. In a separate variant, the recovering step may also involve adding an antisolvent to reduce solubility of the compound and to cause its precipitation, with subsequent isolation of a solid product.

When the solvent and antisolvent technique is used, the suitable antisolvents that may be used for solubility reduction include but are not limited to water, saturated hydrocarbons such as n-hexane, n-heptane, cyclohexane, and the like. Mixtures of any of these solvents are also contemplated. Solubility of the compound can also be reduced by lowering the temperature of a solution or mixture with an antisolvent, such as to temperatures from about −20° C. to about 50° C., or from about −10° C. to about 35° C.

The solid thus obtained may be separated by any technique such as decantation, filtration, centrifugation, etc. and then be further dried. It is generally preferred that rapid drying is utilized to provide the amorphous form of solifenacin succinate with desired stability, moisture content and residual solvent characteristics.

The resultant product may be dried using any methods of drying including spray drying, rotational evaporation (such as using a Buchi Rotavapor), agitated thin film drying, spin-flash drying, fluid-bed drying, lyophilization, or other techniques known in the art.

The process may also include further drying of the product obtained from the solution by vacuum drying over a desiccant, such as phosphorous pentoxide (P₂O₅). The product can also be obtained with other drying agents such as potassium carbonate (K₂CO₃), sodium carbonate (Na₂CO₃), silica gel and the like, as will be apparent to the skilled artisan.

The temperatures for the drying of stable amorphous solifenacin succinate may range from about 25° C. to about 100° C., or about 25° C. to about 75° C., lower temperatures being more suitable at reduced pressures.

The starting material for the process may be crude or pure solifenacin or a salt thereof, such as the succinate, obtained by any method known in the art. The starting material for a process may also be in any polymorphic form, such as a crystalline or an amorphous form, or a mixture of amorphous and crystalline forms obtained by any method. Any polymorphic form of solifenacin or its pharmaceutically acceptable salts such as the succinate are acceptable as starting materials. This includes without limitation the polymorphs or pseudopolymorphs such as solvates, hydrates of solifenacin or its pharmaceutically acceptable salts such as succinate, and if any of these is used as the starting material, the final product will be the corresponding stable amorphous form of the compound.

Seeding particles of the amorphous form of solifenacin or its salt may also be used in the process described herein.

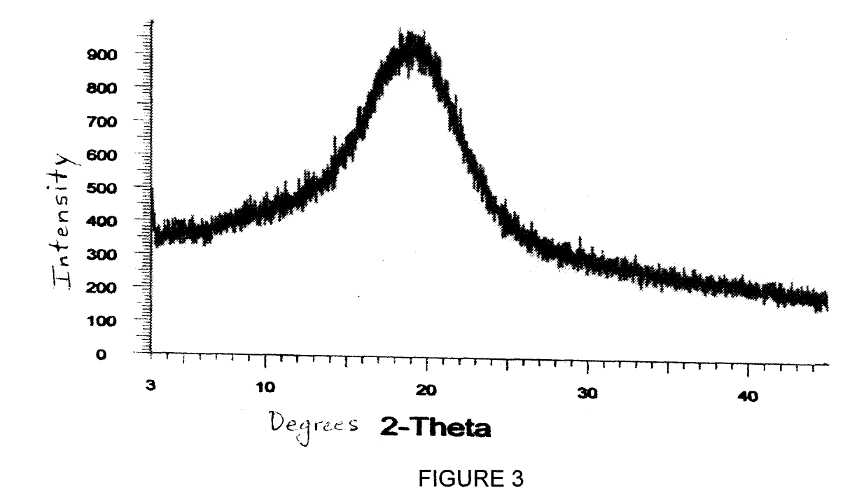

Amorphous solifenacin succinate has been characterized by its X-ray powder diffraction pattern, the patterns described herein being determined on a Bruker AXS D8 Advance powder X-ray diffractometer with a copper K-alpha radiation source. X-ray diffraction patterns were also obtained by methods known in the art using a Bruker X-Ray powder diffractometer, goniometer model 1050/70 at a scanning speed of 1 degree per minute, with copper K-alpha radiation of λ=1.5418 Å. The X-ray diffraction pattern of a sample of amorphous solifenacin succinate is shown as FIG. 3.

In another embodiment, the present invention relates to solid premix compositions of solifenacin or its salts wherein the solifenacin or its salt is present in combination with at least one pharmaceutically acceptable carrier.

Further, the present invention includes processes to prepare premix compositions, wherein an embodiment of a process comprises:

1) providing a solution or dispersion comprising dissolved solifenacin or a salt thereof and at least one pharmaceutical carrier;

2) optionally, filtering a solution; and

3) removing the solvent to recover the premix comprising solifenacin or its salt.

In a further embodiment the present invention includes premix compositions comprising solifenacin succinate in amorphous form.

Also the invention includes stable premix compositions, wherein solifenacin succinate is in an amorphous or substantially amorphous form.

The organic solvents used for dissolving the solifenacin or its pharmaceutically acceptable salts, such as the succinate salt, and the pharmaceutically acceptable carriers and/or crystallization inhibitors can be the same or different solvents can be used.

The products are in the nature of coprecipitates, in which particles of the original components cannot be distinguished using techniques such as microscopy. Without being bound by any specific theory, it is believed that the drug is distributed within the carrier at a molecular level resulting in an amorphous form. Thus, for such a material the energy required for breaking down a crystal structure to bring the drug into solution is reduced, thereby resulting in enhanced solubility, more rapid dissolution, or both. Further, such materials also act as crystallization stabilizers to prevent the conversion of the amorphous form of present invention into other polymorphic forms thus resulting in enhanced stability of the compound at conventional storage temperatures without enhancement in the impurities.

The processing temperature is maintained in a range wherein it does not cause degradation of the product.

The pharmaceutically acceptable carriers that may be used for preparing the compositions or premix compositions include but are not limited to starches, lactose, such as lactose monohydrate, lactose DT, Flowlac™ (available from Meggle Products), Pharmatose™ (available from DMV), mannitol, cellulose derivatives such as hydroxypropyl methylcellulose (HPMC or hypromellose), polymers of N-vinylpyrrolidone commonly known as polyvinylpyrrolidine (“PVP” or “povidone”), powdered celluloses, such Avicel™ PH 101, PH102, PH301, PH302 and PH-F20, microcrystalline cellulose (“MCC”) 102, 114, and 112, silicified microcrystalline cellulose (“SMCC”), such as the PROSOLV™ products sold by JRS Pharma, sorbitol, xylitol, calcium carbonate, magnesium carbonate, dibasic calcium phosphate (Fujicalin®), tribasic calcium phosphate, Veegum™ Zeopharm™ 600 (calcium silicate manufactured by Huber), crospovidone, Neusillin™, croscarmellose sodium, ion exchange resins (for example Amberlite™ IRP 88), zein, colloidal silicon dioxide (Aerosil™), acrylic polymers such as those sold by Evonik Industries as Eudragit™ polymers in different grades such as Eudragit E PO, cyclodextrins or their derivatives, gums, gelatins, hypromellose phthalate, sugars, polyhydric alcohols, polyethylene glycol, polyethylene oxides, polyoxyethylene derivatives, polyvinyl alcohol, propylene glycol derivatives, etc.

Pharmaceutically acceptable hydrophobic carriers include substances such as polyethylene, polybutadiene, polyisoprene, polystyrene, poly(methyl methacrylate) and the like, alkylcelluloses such as natural or synthetic celluloses derivatives (e.g. ethylcellulose), acrylic and methacrylic acid polymers and copolymers, shellac, zein, wax-type substances including hydrogenated castor oil, hydrogenated soyabean oil, hydrogenated vegetable oil, cotton seed oil, or mixtures thereof, acrylic polymers, including but not limited to acrylic acid and methacrylic acid copolymers, methyl methacrylates, methyl methacrylate copolymers, ethoxyethyl methacrylates, cyanoethyl methacrylates, aminoalkyl methacrylate copolymers, poly(acrylic acid), poly(methacrylic acid), methacrylic acid alkylamine copolymers, poly(methylmethacrylate), poly(methacrylic acid)(anhydride), polymethacrylates, polyacrylamides, poly(methacrylic acid anhydride), and glycidyl methacrylate copolymers. This list is not meant to be exclusive, and any pharmaceutically acceptable hydrophobic material, which is capable of stabilizing the amorphous form, may be used in accordance with the present invention.

Melting points for some of the suitable polymeric carriers that are useful in the invention are given in the following table:

| | |
|---|---|
| Povidone K-30 | 300° C. |
| HPMC | Browns at 190-200° C. |
| | and chars at 225-230° C. |
| Methyl cellulose | 240° C. |
| Ethyl cellulose | 240° C. |

Embodiments of the invention include the use of carrier substances that have melting points about 200° C. or higher, and substances such as HPMC that do not melt or decompose below about 200° C.

In embodiments the invention includes stable premix compositions comprising solifenacin succinate and a polyvinylpyrrolidone polymer.

In other embodiments the invention includes stable premix compositions comprising solifenacin succinate and a cellulose derivative such as a hydroxypropyl methylcellulose.

In embodiments the invention includes premix compositions comprising solifenacin or its salts and a resin.

An ion exchange resin (herein after referred as “resin”) is a water-insoluble polymer that contains acidic or basic functional groups and has the ability to exchange counter-ions with aqueous solutions surrounding them. When a drug is loaded onto or released from a resin, a drug ion and an inorganic ion are exchanged. This property allows drugs to be loaded onto resins (forming drug resinates) and then be released in vivo by the salts present in gastrointestinal fluids. A drug-resin complex (“resinate”) possesses physical properties similar to those of the resin. Drug release and physical properties can be manipulated to create many variations of use.

Some of the resins swell significantly on exposure to water. This has led to their use as very effective tablet disintegrants. Further, since the rate of release from a resinate is faster than the rate of the pure drug, resinates can also improve the dissolution characteristics of poorly soluble drugs.

Because resinates are insoluble in water they have no taste. This makes them excellent candidates for taste masking bitter-tasting drugs. As it is known that solifenacin or its salts have a bitter and astringent taste, compositions and formulations with ion exchange resin would mask this undesirable taste.

In many cases, the processes for making a resinate comprise dissolving a drug in a suitable solvent and then adding the resin. Drug loading can take place at ambient temperature and usually takes a few hours to complete. The resinate, so formed, is then isolated either by filtration or by techniques such as spray-drying. In certain cases, for example when resinates are in suspension form, it may not be necessary for resinate to be isolated.

A resin is an insoluble matrix (or support structure) normally in the form of small (e.g., 1-2 mm diameter) beads, usually white or yellowish, fabricated from an organic polymer substrate. There are multiple different types of ion exchange resins which are fabricated to selectively prefer one or several different types of ions.

Ion exchange resins have been classified based on the charge on the exchangeable counter ion (cation exchangers or anion exchangers) and the ionic strength of the bound ion (strong exchangers or weak exchangers). Thus, there are four primary types of ion exchange resins:

1) Strong cation exchange resins, containing sulfonic groups or the corresponding salts.

2) Weak cation exchange resins, containing carboxylic acid groups or the corresponding salts.

3) Strong anion exchange resins containing quaternary ammonium groups. Of these there are two types: Type I resins contain trialkyl ammonium chloride or hydroxide; and Type II resins contain dialkyl 2-hydroxyethyl ammonium chloride or hydroxide.

4) Weak anion exchange resins, containing ammonium chloride or hydroxide.

The resins herein used may be natural, semi-synthetic or synthetic resins, which may be either thermoplastic or thermosetting resins. Suitable ion exchange resins generally have acrylic, methacrylic, phenol-formaldehyde or dextran matrixes.

Different grades of cationic ion exchange resin are available, for example:

1) Amberlite® (sulfonic acid functionality) available grades are IR-120 plus (H), IRP-69, 15, 1200(H), Amberlite® (carboxylic acid functionality) available grades are CG-50 Type I, IRC-50, IRC-505, IRP-88, IRP-64.

2) Dowex® (sulfonic acid functionality) available grades are 50WX2-100, -200, and -400, 50WX4-50, -100, -200, -200R, and -400, 50WX8-100, -200, and -400, HCR-S, HCR-W2,-88, and -650C, MARATHON C, MSC-1, and Dowex® (carboxylic acid functionality) available grade CCR-3.

3) Duolite(sulfonic acid functionality) available grade is C-26.

Different grades of anionic ion exchange resin are available, for example:

1) Amberlite® (trialkylbenzyl ammonium functionality) available grades are IRA-400(CI), -743, and -900, 4200(CI). Amberlite® (dimethyl-2-hydroxyethylbenzyl ammonium functionality) available grade is IRA-410. Amberlite® (polyamine functionality) available grade is IRA-67.

2) Dowex® (trimethylbenzyl ammonium functionality) available grades are 1X2-100, 200, 400; 1X4-50, -100, -200, -400, 1X8-50, -100,-200, -400, MSA-1, 21 K, 550A, Marathon A. Dowex® Type II (dimethyl-2-hydroxyethylbenzyl ammonium functionality) available grades are 2X8-100, -200, -400, MSA-2, Marathon A2, 22. Dowex® (polyamine functionality) available grade is WGR-2, 66, Marathon WBA.

3) Duolite® (polyamine functionality): available grade is A-7.

Mixed bed resins on polystyrene are available, such as Dowex® MR-3, MR-3C, 11A8 Retardation, and also chelating resins for example Amberlite® with iminodiacetic acid exchanger such as IRC-718.

Amberlite and Duolite are trademarks of Rohm and Haas Co. Dowex is a trademark of Dow Chemical Co.

Typical examples of resins include but are not limited to polyethylenes, polypropylenes, vinyl chloride resins, ABS resins, polyesters, polyvinylidine dichlorides, polyamides, polystyrene, polyacetals, polyvinyl alcohols, polycarbonates, acrylic resins, fluorine plastics, polyurethane elastomers, polyester elastomers, phenolic resins, urea resins, melamine resins, unsaturated polyester resins, epoxy resins, urethane resins, rayons, cuprammonium rayons, acetate resins, natural rubbers, synthetic rubbers and EVA resins. These resins may be used alone or in combination.

The amount of drug bound to the resin is determined by the choice of drug, as well as by the resin employed.

In one embodiment, a resinate can be used as produced, or further formulated into an immediate release or a modified release dosage form.

In an embodiment the invention includes premix compositions comprising solifenacin or its salts and a cyclodextrin or its derivatives.

In an embodiment the invention includes stable cyclodextrin complexes of solifenacin succinate.

As used herein, “cyclodextrin” refers to the natural cyclodextrins, α-cyclodextrin, β-cyclodextrin, and γ-cyclodextrin, and their respective derivatives. Derivatives are typically prepared by modifying the hydroxyl groups located on the exterior or hydrophilic side of the cyclodextrin. The complex can modify the physical characteristics of the complex including the formation and dissociation of the complex.

Any of the natural cyclodextrins can be derivatized, such as derivatives of β-cyclodextrin. Cyclodextrin derivatives include alkylated cyclodextrins, comprising methyl-, dimethyl-, trimethyl- and ethyl-β-cyclodextrins; hydroxy alkylated cyclodextrins, including hydroxymethyl-, hydroxyethyl-, hydroxypropyl-, and dihydroxypropyl-β-cyclodextrins, including 2-hydroxypropyl-β-cyclodextrin and 3-hydroxypropyl-β-cyclodextrin, ethylcarboxymethyl cyclodextrins, sulfonate and sulfoalkyl cyclodextrins, such as β-cyclodextrin sulfate, β-cyclodextrin sulfonate, and β-cyclodextrin sulfobutyl ether and 2-hydroxymethyl-β-cyclodextrin sulfate, as well as polymeric cyclodextrins. Other cyclodextrin derivatives can be made by substitution of the hydroxy groups with saccharides, such as glucosyl- and maltosyl-β-cyclodextrin.

Any of the above cyclodextrins or their derivatives or polymers prepared from them can be used for preparation of the premix compositions of the invention, either alone or in the form of mixtures of one or more cyclodextrins.

Commercially available cyclodextrins may be used such as available from any of the commercial suppliers such as for example Cargill, Roquette, Aldrich Chemical Company, Milwaukee Wis. USA, and Wacker Chemicals, New Canaan, Conn. USA, or may be synthesized by any of the processes known in the art for the synthesis of cyclodextrins and their derivatives.

In yet another embodiment the invention includes orally-disintegrating compositions and/or formulations of solifenacin succinate wherein compositions and/or formulations comprise cyclodextrin complexes of solifenacin succinate.

The use of mixtures of more than one of pharmaceutical carrier to provide desired release profiles or for the enhancement of stability is within the scope of this invention. Also, all viscosity grades, molecular weights, commercially available products, their copolymers, and mixtures are all within the scope of this invention without limitation.

In an embodiment the invention includes weight ratios of drug compound to pharmaceutically acceptable carrier or mixture of carriers in the in the range of from about 1:0.1 to about 1:25, or from about 1:1 to about 1:15, or from about 1:1 to about 1:10.

In another embodiment the invention includes premix compositions or formulations, which mask the bitter taste of the drug solifenacin succinate.

In an embodiment the invention includes stable premix compositions of solifenacin succinate comprising at least one pharmaceutically acceptable additive.

The pharmaceutically acceptable additives that can be used for the preparation of stable amorphous form of solifenacin succinate include but are not limited to antioxidants. Some examples of useful antioxidants are butylated hydroxyanisole, butylated hydroxytoluene, ascorbic acid or a salt thereof (e.g., a sodium salt, a calcium salt, a magnesium salt, a potassium salt, a basic amino acid salt, or a meglumine salt), sodium nitrite, sodium hydrogen sulfite, sodium sulfite, a salt of edetic acid (e.g. a sodium salt, a potassium salt, or a calcium salt), erithorbic acid, cysteine hydrochloride, citric acid, cysteine, potassium dichloroisocyanurate, sodium thioglycolate, thioglycerol, sodium formaldehyde sulfoxylate, sodium pyrosulfite, and 1,3-butylene glycol, propyl gallate, and a tocopherol or its derivative. Other antioxidants or chelating agents and such other additives as desired to enhance the stability of the amorphous form of solifenacin succinate are included within the scope of this invention without limitation.

In embodiments the invention includes stable premix compositions comprising solifenacin succinate and at least one antioxidant.

In embodiments the invention includes stable premix compositions comprising solifenacin succinate, at least one pharmaceutically acceptable carrier, and at least one antioxidant.

In embodiments the invention includes the use of weight ratios of solifenacin or its salts to antioxidant in the range of about 1:0.001 to 1:1, or from about 1:0.001 to about 1:0.1.

Processes of the present invention for making stable amorphous solifenacin succinate also include any one or more of mechanical, thermal and solvent processing steps. Exemplary mechanical processing steps include milling and extrusion, melt processing steps include high temperature fusion, solvent-modified fusion and melt-congealing, and solvent processing steps include precipitation, spray coating and spray-drying.

The amorphous form obtained is further dried to remove residual solvents using suitable drying processes, such as tray drying, fluid bed drying, microwave drying, belt drying, rotary drying, vacuum drying, and other drying processes known in the art.

The term “spray-drying” is used conventionally and broadly refers to processes involving breaking up liquid mixtures into small droplets (atomization) and rapidly removing solvent from the mixture in a spray-drying apparatus where there is a strong driving force for evaporation of solvent from the droplets.

The strong driving force for solvent evaporation is generally provided by maintaining the partial pressure of solvent in the spray-drying apparatus well below the vapor pressure of the solvents or mixture of solvents at the temperature of the drying droplets. This may be accomplished by:

(1) maintaining a pressure in the spray-drying apparatus at a partial vacuum (e.g., about 0.01 to about 1 atmosphere, or about 0.01 to about 0.5 atmospheres);

(2) mixing the liquid droplets with a warm drying gas; or

(3) both of (1) and (2).

In addition, at least a portion of the heat required for evaporation of solvent may be provided by heating the sprayed solution.

The spray solution can be delivered to the spray nozzle or nozzles at a wide range of temperatures and flow rates. Generally, the spray solution temperature can range from just above the solvent freezing point to about 20° C. above its ambient pressure boiling point (by pressurizing the solution). Spray solution flow rates to the spray nozzle can vary over a wide range depending on type of nozzle, spray-dryer size and spray-dry conditions such as the inlet temperature and flow rate of the drying gas.

Generally, the energy for evaporation of solvent from the spray solution in a spray-drying process comes primarily from the drying gas.

The drying gas can, in principle, be essentially any gas, but for safety reasons and to minimize undesirable oxidation of the drug or other materials in the solid amorphous dispersion, an inert gas such as nitrogen, nitrogen-enriched air or argon is utilized. The drying gas is typically introduced into the drying chamber at temperatures between about 25° C. and about 100° C.

The amorphous dispersion is usually in the form of small particles. The volume mean size of the particles may be less than about 500 μm, less than about 100 μm, less than about 50 μm, or less than about 25 μm.

When the amorphous form is obtained by spray-drying, the resulting product is in the form of small particles. When the amorphous form is formed by other methods such by melt-congealing or extrusion processes, the resulting solid may be sieved, ground, or otherwise processed to yield a plurality of small particles.

In another embodiment, the invention includes crystalline solifenacin succinate substantially free of amorphous solifenacin succinate and processes to prepare the same.

In an embodiment the present invention includes processes to prepare crystalline solifenacin succinate, wherein an embodiment of a process comprises:

1. providing a solution of solifenacin free base;

2. adding succinic acid to the solution;

3. isolating solifenacin succinate from the solution of step 2; and

4. optionally, drying the obtained solid.

Any of solvents disclosed above may be used for dissolving solifenacin.

Optionally, the solution obtained above can be filtered to remove undissolved particles before further processing, including operations such as, but not limited to, filtration, centrifugation, decantation, and other techniques.

The solution can be filtered by passing it through paper, glass fiber or other membrane materials, or a bed of a clarifying agent such as celite. Depending upon the equipment, concentration and temperature of the solution, the filtration apparatus may need to be preheated to avoid premature crystallization.

Suitably, 1 to 1.5 molar equivalents of succinic acid per equivalent of the starting solifenacin free base are added to the solution obtained from step 1.

Succinic acid can be added at temperatures as high as about 30° C. to about 60° C., or addition can be done at lower temperatures in the range of about 0° C. to about 30° C. The drying can be carried out at reduced pressures, such as below or about 200 mm Hg, or about 50 mm Hg, and at temperatures in the range of about 25° C. to about 80° C., or about 35° C. to about 70° C.

The drying can be carried out for any desired time period for achieving the desired result, such as in the range of about 1 to 20 hours, or longer. Drying may also be carried out for shorter or longer periods of time depending on the product specifications.

The product obtained from the above steps can further be purified by recrystallization or slurrying in a suitable solvent.

In an embodiment the invention includes compositions comprising crystalline solifenacin succinate substantially free of amorphous solifenacin succinate and processes to prepare the same.

The premix compositions can prepared using pharmaceutically acceptable carriers such as polyvinylpyrrolidone, cellulose derivatives such as hydroxypropyl methylcellulose, dibasic calcium phosphate, cyclodextrins or derivatives thereof, resins, and combinations thereof, and may further be formulated into pharmaceutical formulations.

In another embodiment the invention includes pharmaceutical formulations in the form of solid oral dosage forms.

In an embodiment the invention includes pharmaceutical formulations comprising cyclodextrin and solifenacin succinate, wherein a formulation is an orally disintegrating formulation.

In an embodiment the invention includes compositions and/or formulations wherein a composition and/or formulation of solifenacin or its salts is an immediate release form or a modified release form.

In embodiments, formulations comprise solifenacin succinate as an active substance together with at least one of pharmaceutically acceptable excipients such as diluents, disintegrants, binders, glidants, lubricants, antioxidants, sweeteners, flavoring agents, coloring agents, film-forming agents, plasticizers, polishing agents, etc.

The antioxidants as described above for the preparation of premix compositions may also be used for preparing stable pharmaceutical formulations.

In an embodiment the invention includes stable formulations comprising solifenacin succinate and at least one antioxidant.

### Diluents:

Various useful fillers or diluents include but are not limited to starches, lactose, mannitol, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate, hydrogenated castor oil, hydrogenated vegetable oil, hydrogenated soyabean oil, soyabean lecithin, polysorbate 80, cotton seed oil, groundnut oil, soyabean oil or sunflower oil, a mineral oil (for example a paraffin), and an animal oil. It can consist of one or more medium-chain triglycerides. The expression “medium-chain” used here with reference to triglycerides means a linear or branched chain preferably comprising between 8 and 12 carbon atoms approximately. Needless to say, it is possible to use one or more triglycerides in combination. A medium-chain triglyceride used in the composition of the invention can be, for example, a fractionated coconut oil.

### Binders:

Various useful binders include but are not limited to hydroxypropyl cellulose (Klucel™ LF), hydroxypropyl methylcellulose or hypromellose (Methocel™), polyvinylpyrrolidone or povidone (PVP-K25, PVP-K29, PVP-K30, PVP-K90), Plasdone™ S 630 (copovidone), powdered acacia, gelatin, guar gum, carbomer (e.g. carbopol), methylcellulose, polymethacrylates, and starch.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not limited to crosslinked povidone, Kollidon™ CL manufactured by BASF (Germany), Polyplasdone™ XL, XI-10, and INF-10 manufactured by ISP Inc. (USA), and low-substituted hydroxypropylcellulose. Examples of low-substituted hydroxypropylcellulose include but are not limited to low-substituted hydroxypropylcellulose LH11, LH21, LH31, LH22, LH32, LH2O, LH30, LH32 and LH33 (all manufactured by Shin-Etsu Chemical Co., Ltd.). Other useful disintegrants include sodium starch glycolate, colloidal silicon dioxide, and starch.

### Glidants:

Various useful glidants or anti-sticking agents include, but are not limited to talc, silica derivatives, colloidal silicon dioxide and the like and mixtures thereof.

### Lubricants:

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, castor oils, and waxes.

### Colourants:

Colouring agents can be used to colour code the composition, for example, to indicate the type and dosage of the therapeutic agent therein. Suitable colouring agents include, without limitation, natural and/or artificial compounds such as FD & C colouring agents, Food Yellow No. 5, Food Red No. 2, Food Blue No. 2, and the like, food lake colourants, natural juice concentrates, pigments such as titanium oxide, silicon dioxide, and zinc oxide, iron oxides, combinations thereof, and the like.

### Sweeteners:

Useful sweeteners include, but are not limited to, sugars such as sucrose, glucose (corn syrup), dextrose, invert sugar, fructose, and mixtures thereof, acid saccharin and its various salts such as the sodium or calcium salt, cyclamic acid and its various salts such as the sodium salt, the dipeptide sweeteners such as aspartame and alitame, natural sweeteners such as dihydrochalcone compounds, glycyrrhizin, Stevia rebaudiana (stevioside), sugar alcohols such as sorbitol, sorbitol syrup, mannitol (Pearlitol™ SD200), xylitol and the like, synthetic sweeteners such as acesulfame-K and sodium and calcium salts thereof and other synthetic sweeteners, hydrogenated starch hydrolysate (lycasin), protein based sweetening agents such as talin (thaumaoccous danielli), and/or any other pharmacologically acceptable sweetener known in the art, and mixtures thereof.

Suitable sugar alcohols useful as sweeteners include, but are not limited to, sorbitol, xylitol, mannitol (Pearlitol SD200), galactitol, maltitol, isomalt (PALATINIT™) and mixtures thereof. The exact amount of sugar alcohol employed is a matter subject to such factors as the degree of cooling effect desired.

### Flavoring Agents:

Flavoring agents can be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like.

### Film-Forming Agents:

Various useful film-forming agents include but are not limited to cellulose derivatives such as soluble alkyl- or hydroalkyl-cellulose derivatives such as methyl cellulose, hydroxymethyl cellulose, hydroxyethyl cellulose, hydroxypropyl cellulose, hydroxymethyethyl cellulose, hydroxypropyl methylcellulose, sodium carboxy methylcellulose, etc., acidic cellulose derivatives such as cellulose acetate phthalate, cellulose acetate trimellitate and methyl hydroxypropylcellulose phthalate, polyvinyl acetate phthalate, etc., insoluble cellulose derivative such as ethyl cellulose and the like, dextrins, starches and starch derivatives, polymers based on carbohydrates and derivatives thereof, natural gums such as gum Arabic, xanthans, alginates, polyacrylic acid, polyvinyl alcohol, polyvinyl acetate, polyvinylpyrrolidone, polymethacrylates and derivatives thereof (e.g., Eudragit™ products), chitosan and derivatives thereof, shellac and derivatives thereof, and waxes and fat substances.

### Plasticizers:

Various plasticizers for films include but are not limited to castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer used depends upon the type of coating agent.

### Polishing Agents:

Polishing agents that can be used include polyethylene glycols of differing molecular weights and mixtures thereof, talc, surfactants (e.g. glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax). In certain embodiments, polyethylene glycols having molecular weights of 3,000-20,000 are employed.

### Adjuvants:

An opacifier like titanium dioxide may also be used in an amount ranging from about 10% (w/w) to about 20% (w/w) based on the total weight of the coating. Anti-adhesives are frequently used in the film coating process to avoid sticking effects during film formation and drying. A commonly used anti-adhesive for this purpose is talc.

Solvents and antioxidants discussed above as useful to prepare the premix compositions may also be used in processes to prepare pharmaceutical formulations.

As alternatives to the above coating ingredients, pre-formulated commercial coating products such as OPADRY™ (supplied by Colorcon) can conveniently be employed. These products are available from various suppliers and the dry forms require only mixing with a liquid before use.

The formulations of the present invention can be prepared using any processing operations, such as for example one or more of direct compression, dry granulation and wet granulation. Further, a wet granulation method may be conducted using either aqueous or non-aqueous solvents.

In an embodiment the invention includes processes to prepare pharmaceutical formulations of the present invention, wherein an embodiment of a process comprises:

1) sifting solifenacin succinate and excipients such as diluents, disintegrants, binders, glidants, lubricants, etc. through a sieve;

2) dry mixing sifted ingredients;

3) optionally granulating the step 2) materials using binder solution or dispersion and subsequently drying and sizing through a sieve;

4) optionally compacting the step 2) materials into compacts and subsequently milling and sizing through a sieve;

5) placing either step 2) or step 3) or step 4) product into a suitable blender, adding sifted glidants and other excipients, if any, to the blender and blending;

6) adding sifted lubricant to step 5) materials and blending;

7) filling the step 6) product into capsules or compressing into tablets.

The premix compositions or formulations are further characterized for physical parameters such as particle size distribution, bulk density, tap density, moisture content, etc.

An important physicochemical characteristic of particulate compositions is the density properties. Bulk density is described as untapped or tapped. Untapped bulk density of a substance is the undisturbed packing density of that substance and tapped bulk density relates to the packing density after tapping a bed of substance until no change in the packing density is seen. Bulk density and tapped density can be determined using a compendial bulk density apparatus, a suitable method being given in United States Pharmacopeia 29, United States Pharmacopeial Convention, Inc., Rockville, Md., 2005, at pages 2638-2639.

In an embodiment the present invention provides pharmaceutical compositions comprising solifenacin succinate, wherein the bulk density of the final blend ranges from about 0.3 g/ml to about 0.6 g/ml and the tapped density ranges from about 0.4 g/ml to about 0.8 g/ml.

Equipment suitable for processing a pharmaceutical composition of the present invention to produce pharmaceutical formulations include rapid mixer granulators, planetary mixers, mass mixers, ribbon mixers, fluid bed processors, mechanical sifters, blenders, roller compacters, compression machines, rotating bowls or coating pans, tray dryers, fluid bed dryers, rotary cone vacuum dryers, and the like, multi-mills, fluid energy mills, ball mills, colloid mills, roller mills, and hammer mills, and the like.

The dosage forms prepared as above can be subjected to an in vitro dissolution evaluation according to Test 711 “Dissolution” in United States Pharmacopoeia 29, United States Pharmacopeial Convention, Inc., Rockville, Md., 2005 (“USP”), to determine the rate at which the drug substance is released from the dosage forms, and content of drug substance can be determined in solutions by techniques such as high performance liquid chromatography (HPLC).

The pharmaceutical dosage forms of the present invention are intended for oral administration to a patient in need thereof.

Having described the invention with reference to certain embodiments, other embodiments will become apparent to one skilled in the art from consideration of the specification. Certain specific aspects and embodiments of the invention will be further described in the following examples, which are provided solely for purposes of illustration and are not intended to limit the scope of the invention in any manner.

### Example 1

### Preparation of Solifenacin Succinate

### STEP 1: PREPARATION OF N-PHENETHYLBENZAMIDE

Sodium carbonate (0.88 Kg) and water (10 L) were charged into a reactor and stirred for 5 minutes. Phenethylamine (1.0 Kg) was charged into the reactor and the reaction mass was stirred for 10 minutes at 29.8° C. Benzoyl chloride (1.28 Kg) was slowly added to the reaction mass over 1 hour, 50 minutes at 17.5-26.5° C. and the reaction mass was stirred at 21.6-26.6° C. for 2 hours, 30 minutes. The reaction mass was filtered and the solid washed with water (5 L). The product was dried in an air tray dryer at 47.5-56.5° C. for 7 hours, 30 minutes (until the moisture content was less than 1%). Yield: 97.5%.

### STEP 2: PREPARATION OF 1-PHENYL-3,4-DIHYDROISOQUINOLINE

N-phenethyl-benzamide (1 Kg) and polyphosphoric acid (4 Kg) were charged into a reactor. The reaction mass was heated to 160.9° C. and maintained at 160-165° C. for 4 hours, 10 minutes. The reaction mass was cooled to 66.1° C. Water (2 L) was slowly added to the reaction mass at 63-73.5° C. Reaction mass was stirred for 5 minutes, transferred into another reactor and water (13 L) was added at 60-70° C. Reaction mass was cooled to 34.3° C., filtered and the unwanted solid washed with water (1 L). Filtrate was charged into a reactor and cooled to 14.9° C. pH of the reaction mass was adjusted to 2.1 with aqueous sodium hydroxide solution (3.5 Kg NaOH in 5 L water). Toluene (10 L) was added to the reaction mass and pH adjusted to 7.12 with sodium hydroxide solution at 28 to 34° C. Reaction mass was heated to 42° C. and stirred for 10 minutes. Separated the aqueous and organic layers and the aqueous layer was extracted with toluene (5 L). Combined organic layers were washed with water (2×5 L). Solvent was distilled completely under vacuum below 80° C. to get the crude product (Toluene content ≦5% and water content ≦1%). Yield: 77.3%.

### STEP 3: PREPARATION OF 1-PHENYL-1,2,3,4-TETRAHYDRO-ISOQUINOLINE

Methanol (4 L, moisture content ≦0.5%) and 1-Phenyl-3,4-dihydroisoquinoline (1 Kg) were charged into a reactor. The contents were stirred for 10 minutes. Sodium borohydride (0.18 Kg) was added in portions over 1 hour, 45 minutes at 24-29.2° C. Reaction mass was maintained for 2 hours, 30 minutes at 28 to 34° C. Water (10 L) was charged into the reactor at 20-30° C. The contents were stirred for 60 minutes at 28-30° C. The solid was filtered and washed with water (2.5 L). The compound was dried in an air tray dryer at 50-55° C. for 5 hours, 30 minutes (moisture content ≦1). Yield: 96.8%.

### STEP 4: PREPARATION OF (1S)-1-PHENYL-1,2,3,4-TETRAHYDRO-ISOQUINOLINE USING A COMBINATION OF METHANOL AND ETHYL ACETATE AS SOLVENT

1-phenyl-1,2,3,4-tetrahydroisoquinoline (100 g) was placed into a round bottom flask and methanol (400 mL) was added and stirred for about 5 minutes. The reaction mass was then heated to about 40° C., and D-(−)-tartaric acid (71.6 g) was added. The reaction mass was further heated to about 64° C. and maintained for about 2 hours. The reaction mass was then allowed to cool to about 28° C. and ethyl acetate (200 mL) was added. The reaction mass was maintained at about 28° C. for about 20 minutes, and then filtered. The filtered solid was washed with methanol (100 mL) and the wet solid was dried at about 55° C. for about 1 hour, 20 minutes.

The dry material was placed into a round bottom flask and methanol (270 mL) was added. The reaction mass was heated to about 64° C. and maintained for about 1 hour. The reaction mass was then allowed to cool to about 28° C. and ethyl acetate (136 mL) was added. The reaction mass was maintained at about 28° C. for about 1 hour and the solid was filtered and washed with methanol (68 mL). The wet solid was dried at about 50° C. for about 1 hour. The dry solid was placed into a round bottom flask and water (938 mL) was added. The mixture was stirred for about 10 minutes and the pH of the mixture is adjusted to about 8-9 using 10% aqueous sodium hydroxide solution. The mixture was stirred at about 28° C. for about 1 hour and then filtered. The filtered solid was washed with water (125 mL) and dried at about 53° C. for about 9 hours to get 35.9 g of the title compound. Purity by HPLC: 99.24% by weight. Chiral purity by HPLC: 99.64% by weight.

### STEP 5: RECOVERY OF 1-PHENYL-1,2,3,4-TETRAHYDRO-ISOQUINOLINE FROM MOTHER LIQUORS

(1R)-1-phenyl-1,2,3,4-tetrahydroisoquinoline (25 g) recovered from the filtrate of the resolution step, potassium hydroxide (15.5 g), water (12.5 mL), and dimethyl sulfoxide (50 mL) were placed into a clean and dry round bottom flask and stirred for 5 minutes. The reaction mixture was heated to reflux and maintained for 11 hours, 45 minutes. The reaction mass was cooled to 28° C. and chilled water (375 ml) was added to the reaction mixture and stirred for 35 minutes. The separated solid was then filtered, washed with water (25 mL) and dried at about 55° C. to afford 13 g of the title compound.

### STEP 6: PREPARATION OF 1(S)-PHENYL-3,4-DIHYDRO-1H-ISOQUINOLINE-2-CARBOXYLIC ACID ETHYL ESTER USING TOLUENE AS SOLVENT

(1S)-1-phenyl-1,2,3,4-tetrahydroisoquinoline (50 g) and toluene (500 mL) were placed into a round bottom flask and stirred for about 10 minutes. The reaction mass was then cooled to 0° C. and sodium carbonate (27.8 g) was added. Ethylchloroformate (21.15 mL) was then added at about 2 to 3° C. After completing the addition the reaction mass was allowed to reach about 28° C. and maintained for about 2 hours. Reaction completion was determined using thin layer chromatography. After the reaction was complete, the reaction mass was filtered to remove the unwanted solid and the filter bed was washed with toluene (50 mL). The filtrate was washed with water (660 ml) in 2 equal portions. The organic layer was distilled in a Buchi Rotavapor flask at about 65° C. under vacuum to give 64.6 g of the title compound. Purity by HPLC: 98% by weight. Chiral purity by HPLC: 99.37% by weight.

### STEP 7: PREPARATION OF SOLIFENACIN

1(S)-Phenyl-3,4-dihydro-1H-isoquinoline-2-carboxylic acid ethyl ester (100 g) and toluene (500 mL) were placed into a round bottom flask and heated to about 115° C. Moisture was removed from the reaction mass by azeotropic distillation for about 3 hours. Then the reaction mass was cooled to about 55° C. and (3R)-3-quinuclidinol (54.23 g) was added. The reaction mass was heated to about 115° C. and maintained for about 2 hours, then cooled to about 55° C. and sodium hydride (2.81 g) was added. Again the reaction mass was heated to about 115° C. and maintained for about 4 hours. Solvent (50 mL) was removed from the reaction mass by distillation and fresh toluene (50 mL) was added. The reaction mass was maintained at about 115° C. for about 3 hours, and again solvent (50 mL) was removed from the reaction mass and fresh toluene (50 mL) was added. The reaction mass was maintained at about 115° C. for about 3 hours and again solvent (50 mL) was removed from the reaction mass and fresh toluene (50 mL) was added. The reaction mass was then cooled to about 28° C. and saturated aqueous sodium chloride solution (200 mL) was added. The organic layer was separated and washed with water (400 mL). The organic layer was then extracted with a 20% aqueous hydrochloric acid solution (1000 mL). The aqueous layer was then washed with toluene (100 mL). The aqueous layer was cooled to about 15° C. and the pH was adjusted to 10 using an aqueous 20% sodium hydroxide solution (500 mL). Toluene (500 mL) was added to the aqueous layer and stirred for about 10 minutes. The organic layer was separated and the aqueous layer was extracted with toluene (500 mL). The combined organic layer was washed with water (200 mL) in two equal portions. The organic layer was distilled at about 55° C. to give 115 g of the title compound. Purity by HPLC: 90.71% by weight. Chiral purity: 93.3% by weight.

### STEP 8: PREPARATION OF SOLIFENACIN SUCCINATE

Solifenacin (25 g) and acetone (200 mL) were placed into a round bottom flask and stirred for about 15 minutes at about 28° C. The reaction mass was filtered and the filtrate was placed into a round bottom flask. Succinic acid (8.149 g) was added to the above filtrate under stirring. The reaction mass was then heated to about 60° C. and maintained for about 1 hour. The reaction mass was then cooled to about 10-15° C. and maintained for about 1 hour. The separated solid was filtered and washed with about 25 mL of acetone. The wet solid was placed into a round bottom flask with acetone (200 mL) and heated to about 60° C. The reaction mass was maintained at about 60° C. for about 1 hour and then cooled to about 10° C. The reaction mass was maintained at about 10° C. for about 1 hour. The separated solid was filtered and washed with acetone (25 mL). The wet solid was dried at about 50° C. for about 5 hours to yield 25.7 g of the title compound. Purity by HPLC: 99.78% by weight.

### STEP 9: PURIFICATION OF SOLIFENACIN SUCCINATE

Solifenacin succinate 25 g and acetone (750 mL) were charged into a round bottom flask and heated to reflux. The mass was maintained under reflux for 50 minutes and cooled to 12° C. The mass was maintained at 10-12° C. for 1 hour, then the solid was filtered and washed with acetone (25 mL). The compound was dried for 35 minutes at 28° C. and finally dried at 50-55° C. for 5 hours (yield: 78%). Purity by HPLC: 99.85%.

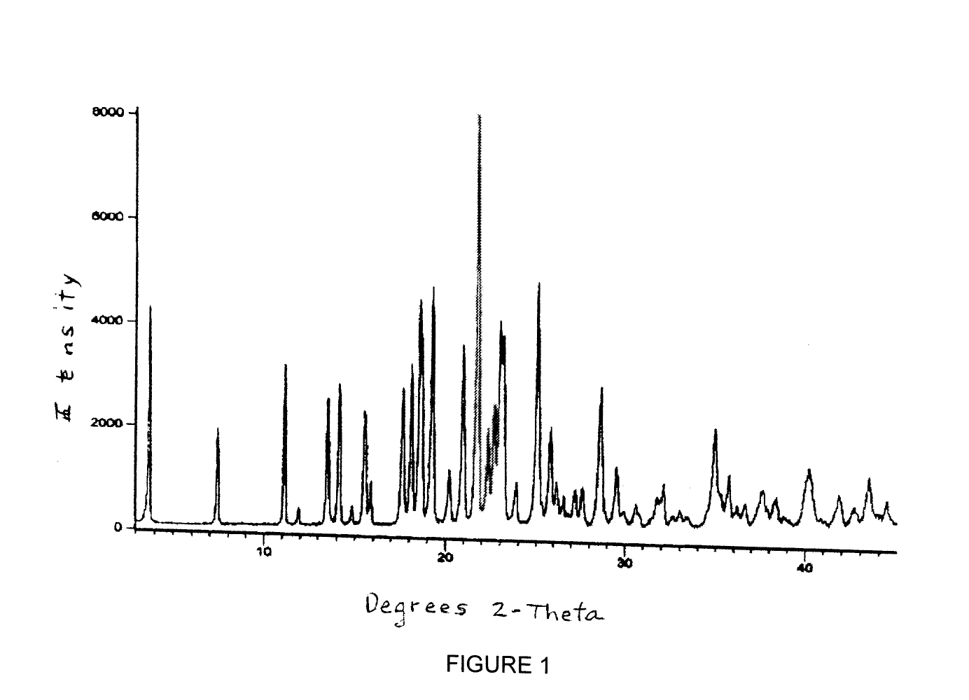

The solid after final drying was analyzed by X-ray powder diffraction (XRD) and the pattern obtained is shown as FIG. 1.

### Example 2

### Amorphous Solifenacin Succinate

| Ingredient | Quantity |
|---|---|
| Solifenacin succinate | 5 g |
| Methanol lot 1* | 60 mL |
| Methanol lot 2* | 5 mL |

*Evaporates during processing.

Manufacturing Process:

1. Methanol lot 1 was charged into a round bottom flask.

2. Solifenacin succinate was added to the step 1 solvent.

3. Step 2 mixtures were continuously stirred for about 10 minutes until it formed a clear solution.

4. The step 3 solutions was filtered and washed with methanol lot 2.

5. Methanol from the step 4 solution was removed by a spray drying process using the following parameters:

Inlet temperature: 75° C.

Outlet temperature: 47-48° C.

Aspirator: about 28 cubic meters per hour.

Pump rate: about 3 mL per minute.

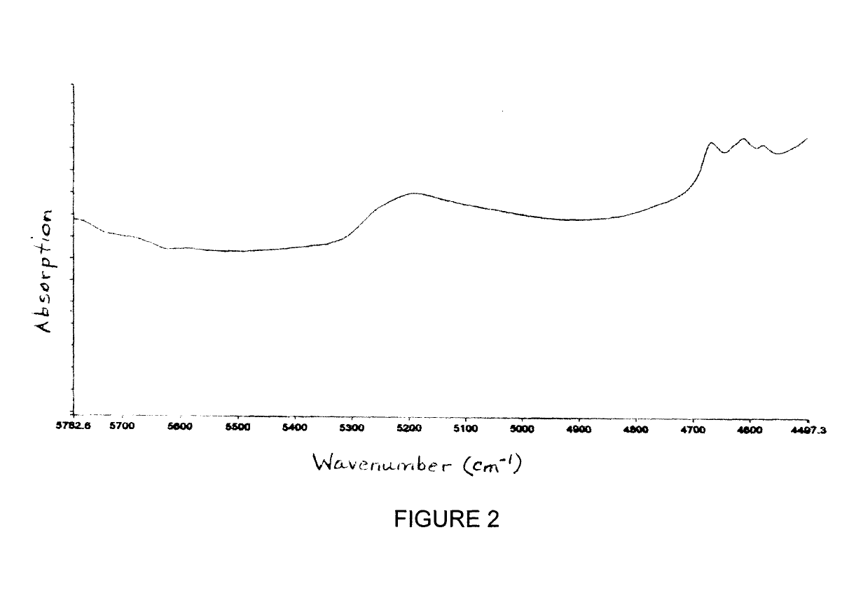

The product was subjected to NIR and XRD analysis and the absorption spectrum and pattern are FIGS. 2 and 3, respectively.

The spray-dried product was packaged in a triple laminated package (two polyethylene layers covered with a layer of aluminium foil) containing a nitrogen atmosphere and samples were analyzed for impurity content at the time of packaging and after 7 days of storage at 0-5° C., using high-performance liquid chromatography (HPLC). The results are tabulated below:

| Parameter | Initial | 7^{th} Day |
|---|---|---|
| HSI* (% of drug content) | 0.03 | 0.25 |

*Highest single impurity.

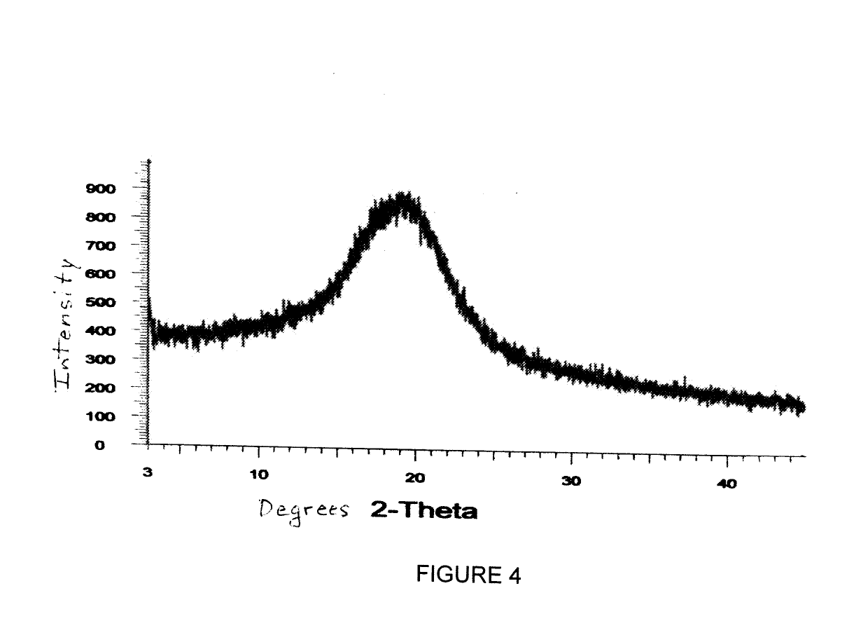

After storage, a sample had the XRD pattern of FIG. 4.

### Example 3

### Premix Composition Comprising Solifenacin Succinate and Povidone

| Ingredient | Quantity |
|---|---|
| Solifenacin succinate | 20 g |
| Povidone K-30 | 20 |
| Methanol lot 1* | 550 mL |
| Methanol lot 2* | 50 mL |

*Evaporates during processing.

Manufacturing Process:

1. Solifenacin succinate and methanol lot 1 were charged into a round bottom flask.

2. The mixture was stirred until it formed a clear solution.

3. Povidone was added to the step 2 solution.

4. The solution of step 3 was filtered through paper and a Hyflow (flux calcined diatomaceous earth) bed filter and was washed with methanol lot 2.

5. The filtrate was placed into a Buchi Rotavapor and rapidly evaporated under vacuum at 60° C.

6. The dried solid was packed in a double polyethylene bag with a silica desiccant pouch and the package was exposed to 0-5° C. and room temperature (RT) conditions for 25 days, then was analyzed by XRD and HPLC. The analytical data are given below:

| Parameter | 0-5° C. | RT |
|---|---|---|
| XRD | Amorphous | Amorphous |
| Drug purity (%) | 99.41 | 99.27 |
| HSI* (% of drug | 0.39 | 0.51 |
| content) | | |

*Highest single impurity.

### Examples 4A and 4B

### Premix Compositions Comprising Solifenacin Succinate with Povidone and Antioxidants

Quantity

| | | |
|---|---|---|
| Ingredient | Example 4A | Example 4B |

| | | |
|---|---|---|
| Solifenacin succinate | 4 | g |
| Povidone | 2 | g |
| Methanol lot 1* | 80 | mL |
| Methanol lot 2* | 10 | mL |

| | | |
|---|---|---|
| Butylated hydroxytoluene | 0.04 g | — |
| (BHT) | | |
| Propyl gallate | — | 0.04 g |

*Evaporates during processing.

Manufacturing process: same as that of Example 3 except that BHT was included in step 1.

The solid prepared was packaged in a double polyethylene bag with a silica desiccant and exposed to 0-5° C. and room temperature (RT) conditions for 27 days, and then samples were analyzed by XRD and HPLC. The data are given below:

| | Example 4A | | Example 4B | |
|---|---|---|---|---|
| Parameter | 0-5° C. | RT | 0-5° C. | RT |
| XRD | Amorphous | Amorphous | Amorphous | Amorphous |
| Drug purity | 99.75 | 99.74 | 99.71 | 99.72 |
| (%) | | | | |
| HSI* (% of | 0.25 | 0.26 | 0.29 | 0.28 |
| drug content) | | | | |

*Highest single impurity.

### Example 5

### Premix Composition of Solifenacin Succinate with Hydroxypropyl Methylcellulose

| Ingredient | Quantity | |
|---|---|---|
| Solifenacin succinate | 1 | g |
| Hydroxypropyl | 0.5 | g |
| methylcellulose 5 cps | | |
| Methanol lot 1* | 25 | mL |
| Methanol lot 2* | 5 | mL |

*Evaporates during processing.

Manufacturing process: same as Example 3.

The dried solid was packaged in a double polyethylene bag with a silica desiccant and exposed to 0-5° C. conditions for 16 days. A sample was then analyzed by XRD and HPLC, giving the following results:

| Parameter | Result |
|---|---|
| XRD | Amorphous |
| Drug purity (%) | 99.9 |
| HSI* (% of drug content) | 0.05 |

*Highest single impurity.

### Examples 6A and 6B

### Premix Compositions of Solifenacin Succinate with HPMC and Antioxidants

Quantity

| | | |
|---|---|---|
| Ingredient | Example 6A | Example 6B |

| | | |
|---|---|---|
| Solifenacin succinate | 4 | g |
| Hydroxypropyl | 2 | g |
| methylcellulose 5 cps | | |
| Methanol* | 240 | mL |

| | | |
|---|---|---|
| Butylated hydroxytoluene | 0.04 g | — |
| (BHT) | | |
| Propyl gallate | — | 0.04 g |

*Evaporates during processing.

Manufacturing process: same as that of Example 3 except that BHT has been included in step 1.

The solid prepared was packaged in a double polyethylene bag and exposed at 0-5° C. and at room temperature (RT) for 20 days. Samples were then analyzed by XRD and by HPLC, giving the data below:

| | Example 6A | | | Example 6B | | |
|---|---|---|---|---|---|---|
| Parameter | Initial | 0-5° C. | RT | Initial | 0-5° C. | RT |
| XRD | Am | Am | Am | — | Am | Am |
| Drug purity (%) | 99.96 | 99.96 | 99.96 | 99.92 | 99.97 | 99.97 |
| HSI* (% of drug | — | 0.04 | 0.04 | — | 0.03 | 0.03 |
| content) | | | | | | |

*Highest single impurity.
Am = amorphous.

The solid products of Examples 6A and 6B, similarly packaged and stored under 0-5° C. and RT conditions for about 7 months, were analyzed and the data are given below:

| | | | Drug Purity | | | |
|---|---|---|---|---|---|---|
| Sample | Storage | RD | (%) | HSI | SOS-4A | TI |
| Example | Initial | Am | 99.96 | — | — | — |
| 6A | 0-5° C. | — | 99.76 | 0.11 | — | 0.24 |
| | RT | — | 99.68 | 0.12 | — | 0.32 |
| Example | Initial | — | 99.92 | — | — | — |
| 6B | 0-5° C. | Am | 99.93 | 0.03 | — | 0.07 |
| | RT | Am | 99.82 | 0.09 | — | 0.18 |

HSI = highest single impurity,
SOS-4A = (1S)-1-phenyl-1,2,3,4-tetrahydro-isoquinoline, and
TI = total impurities (all values expressed as % of drug content).
Am = amorphous.

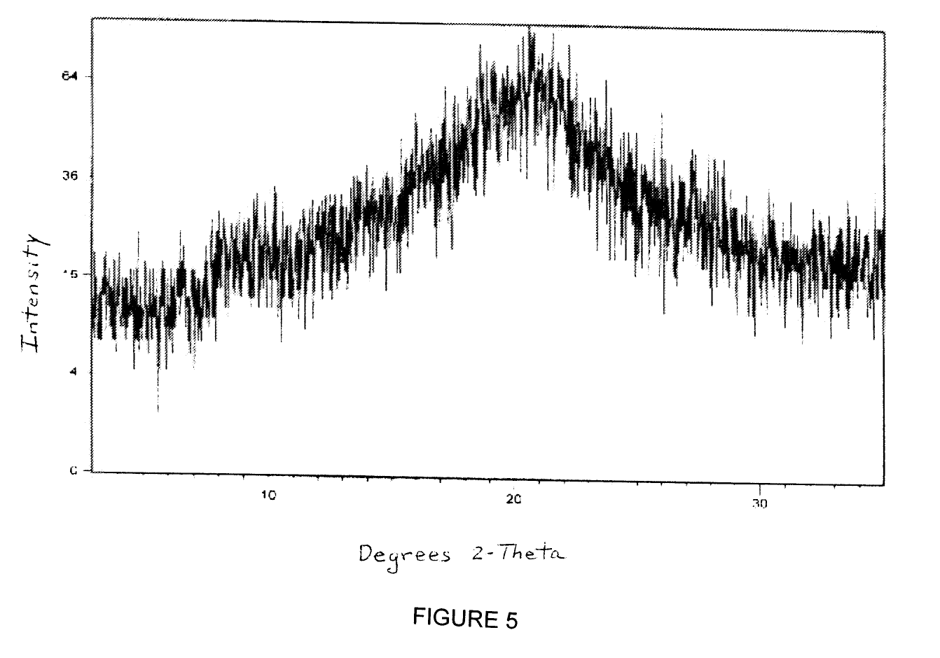

The XRD pattern for the composition of Example 6B, after exposure to RT conditions for 6 months is shown as FIG. 5.

### Example 7

### Premix Composition of Solifenacin Succinate with Ethylcellulose

| Ingredient | Quantity | |
|---|---|---|
| Solifenacin succinate | 1 | g |
| Ethylcellulose (Ethocel ™) | 0.5 | g |
| Methanol lot 1* | 25 | mL |
| Methanol lot 2* | 5 | mL |

*Evaporates during processing.

Manufacturing process: same as Example 3.

### Examples 8A and 8B

### Premix Compositions of Solifenacin Succinate with Ethylcellulose and Antioxidants

Quantity

| | | |
|---|---|---|
| Ingredient | Example 8A | Example 8B |

| | | |
|---|---|---|
| Solifenacin succinate | 4 | g |
| Ethylcellulose (Ethocel) | 2 | g |
| Methanol* | 240 | mL |

| | | |
|---|---|---|
| Butylated hydroxytoluene | 0.04 g | — |
| (BHT) | | |
| Propyl gallate | — | 0.04 g |

*Evaporates during processing.

Manufacturing process: same as that of Example 3 except that BHT has been included in step 1 for Example 8A.

The solid prepared was packaged in a double polyethylene bag and exposed to 0-5° C. and at room temperature (RT) for about 20 days. Samples were analyzed by XRD and HPLC and the data are given below:

| | Example 8A | | | Example 8B | | |
|---|---|---|---|---|---|---|
| Parameter | Initial | 0-5° C. | RT | Initial | 0-5° C. | RT |
| XRD | Am | Am | Am | — | Am | Slight |
| | | | | | | crystallinity |
| Drug purity (%) | 99.75 | 99.91 | 99.94 | 99.92 | 99.96 | 99.92 |
| HSI* (% of drug | — | 0.08 | 0.06 | — | 0.04 | 0.05 |
| content) | | | | | | |

*Highest single impurity.

### Example 9

### Formulation for Solifenacin Succinate 10 mg Tablets

Composition of Solifenacin Succinate Resinate:

| Ingredient | mg/Tablet |
|---|---|
| Solifenacin succinate | 10 |
| Amberlite IRP 88 | 40 |
| Water | 320 |

Manufacturing Process:

1. Amberlite IRP 88 was dispersed in water under stirring to form a suspension.

2. Solifenacin succinate was added to the suspension of step 1 and stirring was continued for about 5.5 hours.

3. The suspension of the step 2 was centrifuged and vacuum filtered.

4. The solifenacin succinate resinate was dried in oven at 60° C. for about 15 hours.

5. The dried solifenacin succinate resinate of step 4 was sifted through a BSS #60 mesh sieve.

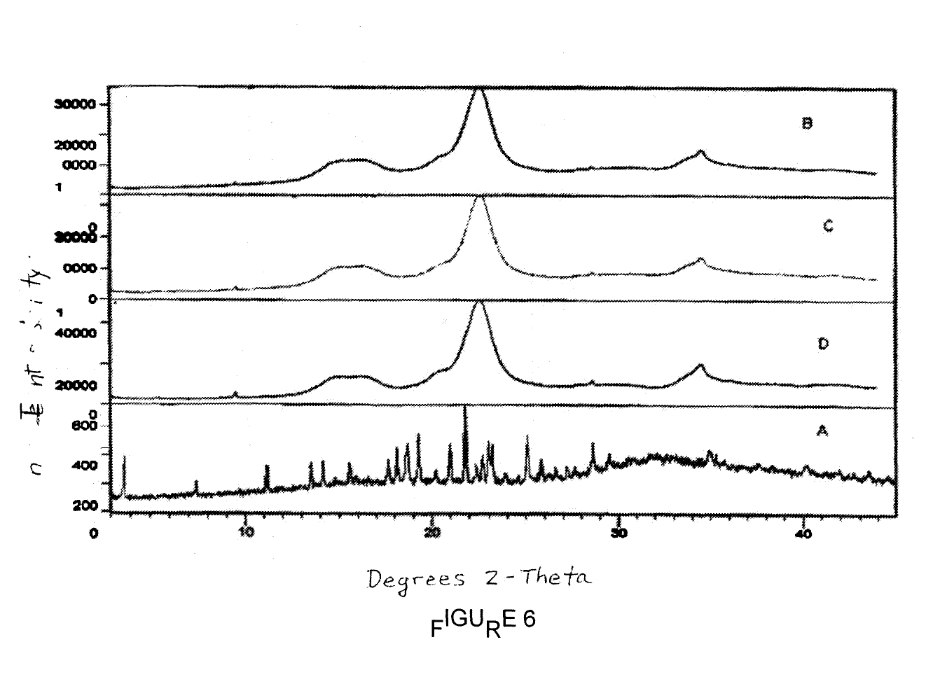

The XRD patterns for solifenacin succinate, a physical mixture of solifenacin succinate and Amberlite IRP 88 in the proportions given above, and solifenacin succinate resinate prepared above are respectively shown in FIG. 6.

Tablet Formulation:

| Ingredient | mg/Tablet |
|---|---|
| Solifenacin succinate resinate | 50 |
| Microcrystalline cellulose (Avicel PH 102) | 197.5 |
| Magnesium stearate | 1.25 |
| Talc | 1.25 |

Manufacturing Process:

1. Avicel PH 102 was sifted through a BSS #30 mesh sieve and blended with the dried solifenacin succinate resinate, prepared above, in a double cone blender for about 20 minutes.

2. Magnesium stearate and talc were sifted through a BSS # 60 mesh sieve, added to step 1, and blended for about 5 minutes.

3. The resulting blend was compressed into tablets using a 8.5 mm round punch.

The tablets were packaged in closed HDPE containers and exposed to 40° C. and 75% RH conditions for 3 months. Testing results are tabulated below:

| Parameter | Initial | 2 Months | 3 Months |
|---|---|---|---|
| XRD | Amorphous | Amorphous | Amorphous |

Dissolution*

| | | | |
|---|---|---|---|
| 10 minutes | 71 | 75 | 87 |
| 20 minutes | 79 | 81 | 91 |
| 30 minutes | 83 | 84 | 94 |
| 45 minutes | 86 | 86 | 97 |
| 60 minutes | 89 | 89 | 98 |

Impurities**

| | | | |
|---|---|---|---|
| SOS-4A | 0.007 | 0.03 | NP |
| HSI*** | 0.05 | 0.1 | NP |
| TI^{‡} | 0.3 | 0.42 | NP |

*Dissolution conditions: 0.1 N HCl, 900 ml, Type II apparatus, 37° C. ± 0.5° C., 50 rpm. Values are cumulative percentages of contained drug that dissolved.
**Values are percentages of the original solifenacin succinate content.
***Highest single impurity.
^{‡}Total impurities.
NP = not performed.

### Examples 10-12

### Formulations of Solifenacin Succinate 10 mg Tablets

| | Quantity | | | | | |
|---|---|---|---|---|---|---|
| Ingredient | Example 10 | | Example 11 | | Example 12 | |
| Solifenacin succinate | 2 | g | 2 | g | 1 | g |
| HPMC 5 cps | 2 | g | — | | — | |
| β-cyclodextrin | — | | — | | 1 | g |
| Eudragit EP O^{‡} | — | | 2 | g | — | |
| Microcrystalline cellulose | 23.6 | g | 23.6 | g | 11.8 | g |
| (MCC PH 102) | | | | | | |
| Croscarmellose sodium | 1.5 | g | 1.5 | g | 0.75 | g |
| (CCS) | | | | | | |
| Talc | 0.3 | g | 0.3 | g | 0.15 | g |
| Colloidal silicon dioxide | 0.3 | g | 0.3 | g | 0.15 | g |
| (Aerosil) | | | | | | |
| Magnesium stearate | 0.3 | g | 0.3 | g | 0.15 | g |

| Isopropyl alcohol (IPA)* | 14.5 | mL | — | | — |
|---|---|---|---|---|---|
| Dicholoromethane (DCM)* | 16.5 | mL | 5 | mL | — |

| | | | | |
|---|---|---|---|---|
| Water* | — | — | 10 | mL |

| | | | | |
|---|---|---|---|---|
| Acetone* | — | 5 | mL | — |

*Evaporates during processing.
^{‡}Eudragit ™ E is a cationic copolymer based on dimethylaminoethyl methacrylate and neutral methacrylates.

Manufacturing Process:

1. Solifenacin succinate and either HPMC 5 cps (Example 10),

Eudragit E PO (Example 11), or β-cyclodextrin (Example 12) were dissolved in the solvents.

2. MCC PH 102 (50% of the total amount) was granulated with the above-prepared solutions and granules were dried at 60° C. for 15 minutes (for Example 10, dried for 2 hours).

3. Remaining MCC and the COS were blended with the dried granules.

4. To the above blend, talc, Aerosil (sifted through a ASTM #40 mesh sieve) and magnesium stearate (sifted through a ASTM #80 mesh sieve) were added and mixed thoroughly.

5. Tablets were compressed using a 7 mm round punch to a weight of 150 mg per tablet using a compression machine.

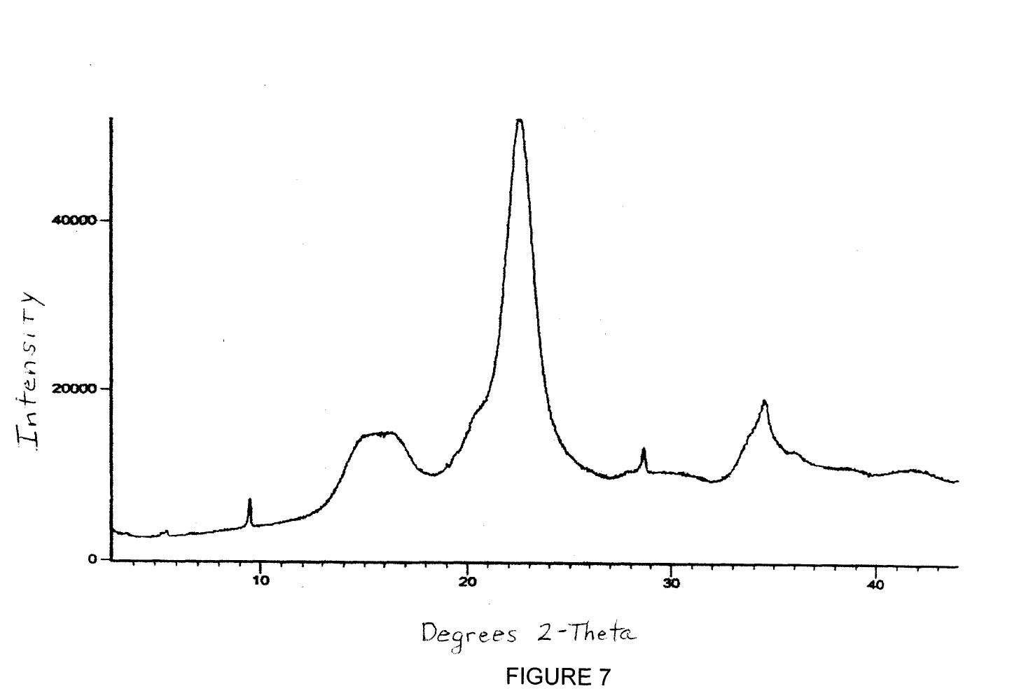

An XRD pattern of the product of Example 10 is shown as FIG. 7.

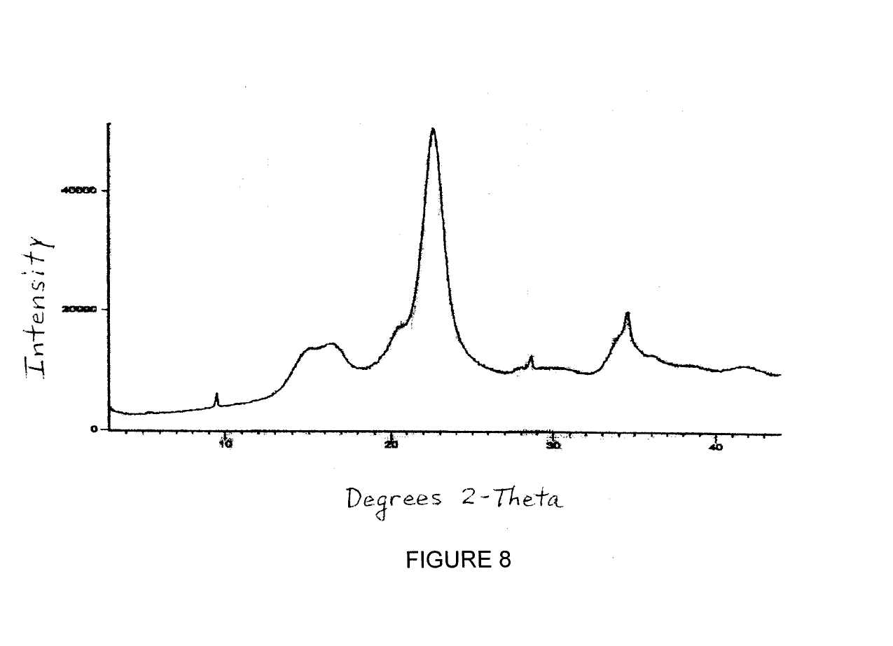

An XRD pattern of the product of Example 12 is shown as FIG. 8.

### Examples 13 and 14

### Solifenacin Succinate 10 mg Tablets Using Zeopharm™ 600

| | mg/Tablet | |
|---|---|---|
| Ingredient | Example 13 | Example 14 |
| Solifenacin succinate | 10 | 10 |
| Propylene glycol | 13.9 | 13.9 |
| Zeopharm ™ 600^{‡} | 15.29 | — |
| Dibasic calcium phosphate (Fujicalin ™) | | 47.8 |
| Croscarmellose sodium | 7.5 | 7.5 |
| Microcrystalline cellulose (MCC PH | 101.82 | 69.3 |
| 102) | | |
| Magnesium stearate | 0.75 | 0.75 |
| Talc | 0.75 | 0.75 |

^{‡}Zeopharm ™ 600 chemically is calcium silicate, manufactured by Huber.

Manufacturing Process:

1. Solifenacin succinate was dissolved in propylene glycol to form a clear solution.

2. Solution of step 1 was adsorbed onto Zeopharm 600 (Example 13) or Fujicalin (Example 14) and mixed thoroughly.

3. MCC PH 102 and COS were sifted through a ASTM 40 mesh sieve.

4. Step 3 materials were added to step 2 materials.

5. The blend of step 4 was placed into a double cone blender and blended for about 10 minutes.

6. Talc and magnesium stearate were sifted through a ASTM 80 mesh sieve, added to the blend of step 5, and blended for about 5 minutes.

7. The final blend of step 6 was compressed into tablets using a compression machine.

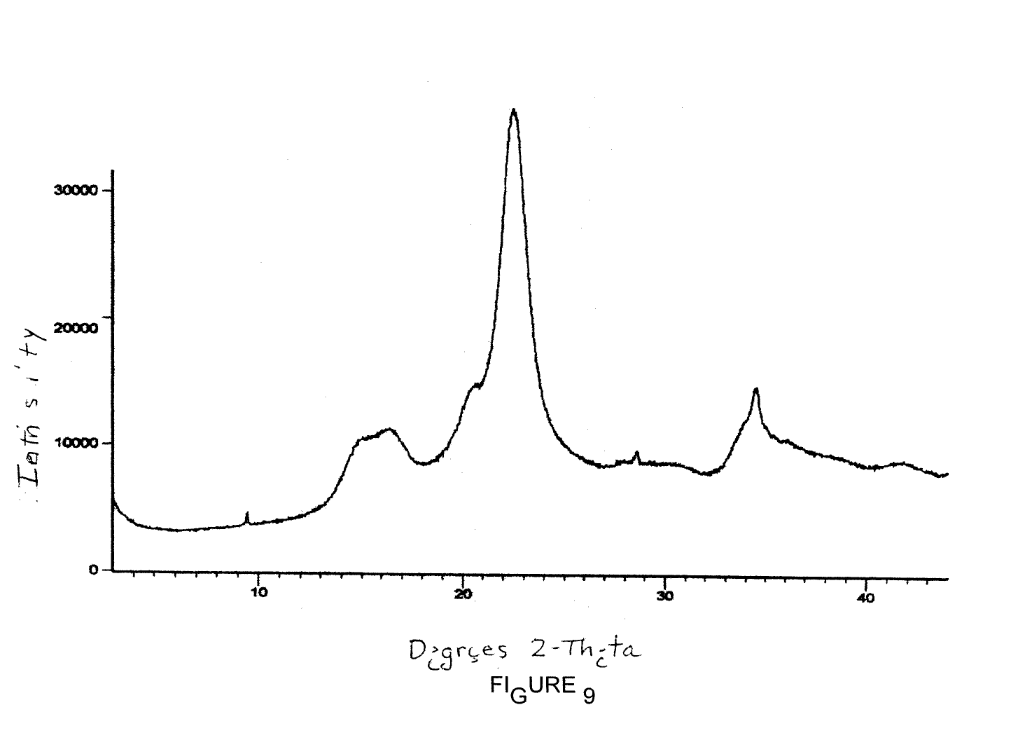

An XRD pattern of the product of Example 13 is shown as FIG. 9.

The tablets of Example 14 were packaged in closed HDPE containers and exposed to accelerated stability testing conditions of 40° C. and 75% RH for 1 month, and analytical data are given below:

| Parameter | Initial | 1 Month |
|---|---|---|
| XRD | Amorphous | Amorphous |

Impurities*

| | | |
|---|---|---|
| SOS-4A | ND | ND |
| HSI** | 0.043 | 0.0406 |
| TI^{‡} | 0.107 | 0.1133 |

*Values are percentages of the original solifenacin succinate content.
**Highest single impurity.
^{‡}Total impurities.
ND = not detected.

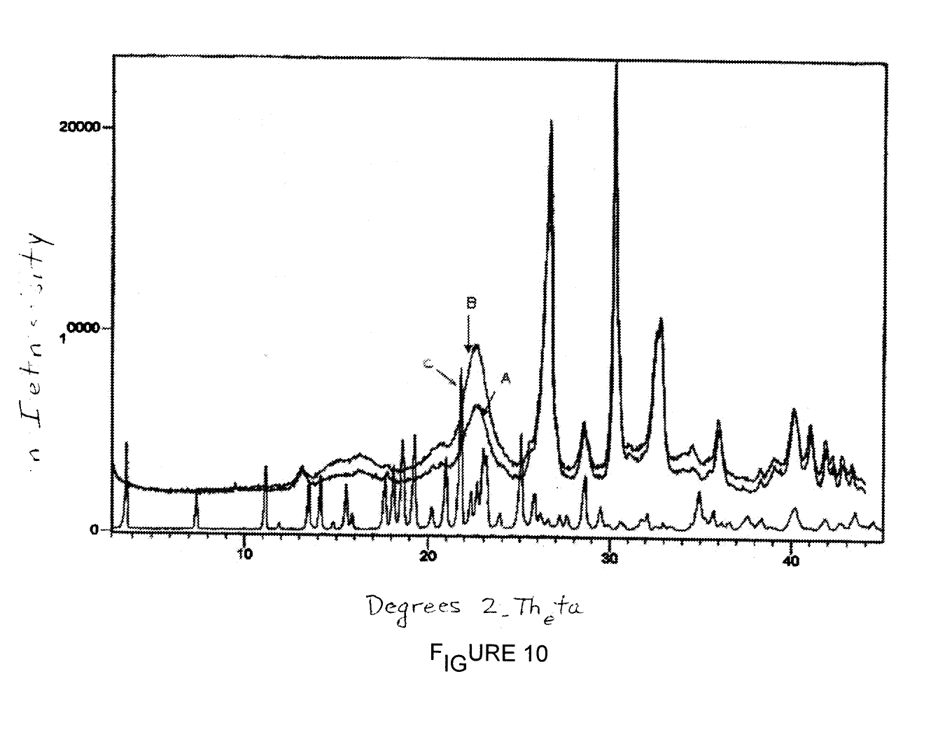

Comparative XRD patterns for the composition prepared according to Example 14 after storage under 40° C. and 75% RH conditions for 3 months (A), the initial composition before exposure (B), and crystalline solifenacin succinate (C) are shown in FIG. 10.

### Example 15

### Formulation of Solifenacin Succinate 10 mg Tablets

| Ingredient | mg/Tablet |
|---|---|
| Solifenacin succinate | 10 |
| Hydroxypropyl-β-cyclodextrin (HPβCD) | 10 |
| Microcrystalline cellulose 102 (Avicel PH 102) | 120.25 |
| Croscarmellose sodium | 7.5 |
| Colloidal silicon dioxide | 0.75 |
| Magnesium stearate | 0.75 |
| Talc | 0.75 |
| Water* | q.s. |

*Evaporates during processing.

Manufacturing process: same as that of Example 12, but β-cyclodextrin was replaced with hydroxypropyl-β-cyclodextrin.

The tablets were packaged in closed HDPE containers and stored under accelerated storage testing conditions of 40° C. and 75% RH for 3 months, and analytical data are given below:

| Parameter | Initial | 1 Month | 2 Months | 3 Months |
|---|---|---|---|---|
| XRD | Amorphous | Amorphous | Amorphous | Amorphous |

Dissolution*

| | | | | |
|---|---|---|---|---|
| 10 minutes | 89 | 99 | 86 | 104 |
| 20 minutes | 97 | 99 | 94 | 106 |
| 30 minutes | 98 | 99 | 96 | 106 |
| 45 minutes | 98 | 99 | 95 | 107 |

Impurities**

| | | | | |
|---|---|---|---|---|
| SOS-4A | ND | 0.01 | ND | ND |
| HSI*** | 0.08 | 0.1 | 0.07 | 0.07 |
| TI^{‡} | 0.23 | 0.37 | 0.27 | 0.33 |

*Dissolution conditions: 0.1 N HCl, 900 ml, Type II apparatus, 37° C. ± 0.5° C., 50 rpm. Values are cumulative percentages of contained drug that dissolved.
**Values are percentages of the original solifenacin succinate content.
***Highest single impurity.
^{‡}Total impurities.
ND = Not detected.

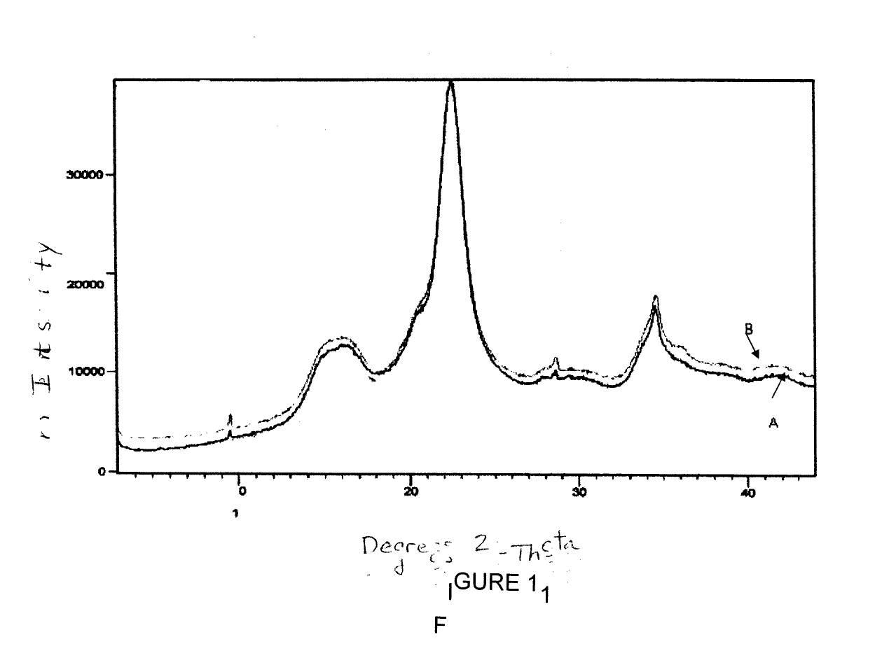

Comparative XRD patterns for the composition prepared according to Example 15 after storage under 40° C. and 75% RH conditions for 3 months (A), and a similarly prepared composition but without solifenacin succinate (B) are shown in FIG. 11.

### Example 16

### Formulation of Solifenacin Succinate 10 mg Capsules

| Ingredient | mg/Capsule |
|---|---|
| Solifenacin succinate | 10 |
| Medium chain triglycerides | 156 |
| Hydrogenated soyabean oil | 38 |
| Soyabean lecithin | 14.5 |
| Polysorbate 80 | 43.5 |

Manufacturing Process:

1. Mix solifenacin succinate with medium chain triglycerides and hydrogenated soyabean oil.

2. To the step 1 mixture add soyabean lecithin and polysorbate 80 and mix homogeneously.

3. Encapsulate the mixture of step 2 into soft gelatin capsules.

### Example 17

### Formulations Containing Solifenacin Succinate 10 mg

| | mg/Capsule |
|---|---|
| Ingredient | or Tablet |
| Solifenacin succinate | 10 |
| Medium chain triglycerides | 156 |
| Hydrogenated castor oil | 38 |
| Methylene chloride* | 16.5 |
| Isopropyl alcohol* | 14.5 |
| Microcrystalline cellulose (MCC PH | 101.82 |
| 102) | |
| Croscarmellose sodium (CCS) | 1.5 |
| Talc | 0.75 |
| Colloidal silicon dioxide (Aerosil) | 0.25 |
| Magnesium stearate | 0.75 |

*Evaporates during processing.

Manufacturing Process:

1. Dissolve medium chain triglycerides and hydrogenated castor oil in the solvent system of isopropyl alcohol and methylene chloride.

2. Dissolve solifenacin succinate in the solution of step 1) with stirring to form a clear solution.

3. Spray dry the step 2) solution to obtain a solid powder.

4. Sift MCC PH 102, COS, and colloidal silicon dioxide through a ASTM #40 mesh sieve.

5. Add step 4 materials to step 3 materials.

6. Place the blend of step 5 into a double cone blender and blend for about 10 minutes.

7. Sift talc and magnesium stearate through a ASTM 80 mesh sieve, add to the blend of step 6, and blend for about 5 minutes.

8. Compress the final blend of step 7 into tablets using a compression machine or fill the blend into capsules.

1. A process for preparing stabilized solifenacin or a salt thereof, comprising:
(a) providing a solution containing solifenacin or a salt thereof, a pharmaceutically acceptable carrier, and optionally an antioxidant, then removing solvent or adding an anti-solvent to precipitate a solid; or
(b) providing a solution containing solifenacin or a salt thereof in a nonvolatile solvent, and optionally adsorbing the solution onto a solid pharmaceutical excipient; or
(c) providing a solution containing solifenacin or a salt thereof, and contacting the solution with an insoluble resin to form a resinate; or
(d) providing a solution containing solifenacin or a salt thereof and a cyclodextrin, and combining the solution with a solid pharmaceutical excipient.

2. The process of claim 1, wherein stabilized solifenacin or a salt thereof is amorphous.

3. The process of claim 1, wherein solifenacin or a salt thereof comprises solifenacin succinate.

4. The process of claim 1, wherein a pharmaceutically acceptable carrier comprises a polymer.

5. The process of claim 1, wherein a pharmaceutically acceptable carrier comprises a polyvinylpyrrolidone polymer or a cellulose derivative.

6. The process of claim 1, wherein a pharmaceutically acceptable carrier comprises a hydroxypropyl methylcellulose.

7. The process of claim 1, wherein an antioxidant comprises one or more of butylated hydroxyanisole, butylated hydroxytoluene, and propyl gallate.

8. The process of claim 1, wherein a weight ratio of solifenacin or its salt to antioxidant is about 1:0.001 to about 1:1.

9. The process of claim 1, wherein a weight ratio of solifenacin or its salt to antioxidant Is about 1:0.001 to about 1:0.1.

10. The process of claim 1, wherein an antisolvent comprises water or a saturated hydrocarbon.

11. A pharmaceutical formulation comprising stabilized solifenacin or a salt thereof prepared by the process of claim 1, and at least one pharmaceutical excipient.

12. A solid premix composition prepared by combining a solution comprising solifenacin succinate and an organic solvent with a pharmaceutically acceptable carrier, and removing solvent.

13. The solid premix composition of claim 12, wherein a pharmaceutically acceptable carrier is a solid, when combined with a solution comprising solifenacin succinate.

14. The solid premix composition of claim 12, wherein a pharmaceutically acceptable carrier is in solution or dissolves, when combined with a solution comprising solifenacin succinate.

15. The solid premix composition of claim 12, wherein a solution further comprises an antioxidant.

16. The solid premix composition of claim 12, in which solifenacin succinate is amorphous.

17. A pharmaceutical formulation comprising a solid premix composition of claim 12, and at least one pharmaceutical excipient.

18. A method for treating an overactive bladder disorder, comprising administering a pharmaceutical formulation of claim 11.

19. A method for treating an overactive bladder disorder, comprising administering a pharmaceutical formulation of claim 17.

20. A pharmaceutical formulation comprising a solid premix composition of claim 16, and at least one pharmaceutical excipient.
